(19) United States
(12) Patent Application Publication
(10) Pub. No.: US 2023/0302033 A1
(43) Pub. Date: Sep. 28, 2023

(54) FORMULATIONS OF NICOTINIC ACID DERIVATIVES AND FLAVONOID POLYPHENOLS AND USES THEREOF

(71) Applicant: Animal Biosciences, Inc., Boston, MA (US)

(72) Inventors: Virginia RENTKO, Boston, MA (US); Nicholas SINCLAIR, Boston, MA (US)

(21) Appl. No.: 18/020,880

(22) PCT Filed: Apr. 13, 2022
(86) PCT No.: PCT/US2022/024604

§ 371 Date: Feb. 10, 2023

Related U.S. Application Data
(60) Provisional application No. 63/310,881, filed on Feb. 16, 2022.
(60) Provisional application No. 63/174,456, filed on Apr. 13, 2021.

(51) Int. Cl. A61K 31/706; A61K 31/352; A61P 39/00

(52) U.S. Cl. CPC A61K 31/706; A61K 31/352; A61P 39/00

(57) ABSTRACT

The present application relates to formulations comprising a combination of an NAD+ agonist and a flavonoid polyphenol. Also provided herein are methods of administering a combination of an NAD+ agonist and a flavonoid for use in methods for treating, ameliorating, or preventing at least one symptom or indication an age-related disease or disorder in a subject.

### CROSS REFERENCE TO RELATED APPLICATIONS

This application claims the benefit of U.S. Provisional Patent Application No. 63/174,456, filed Apr. 13, 2021, and U.S. Provisional Patent Application No. 63/310,881, filed Feb. 16, 2022, the contents of which are incorporated herein by reference in their entirety.

### TECHNICAL FIELD

The field of the invention relates to formulations of nicotinic acid derivatives, formulations of flavonoid polyphenols, processes of preparation and methods of use thereof.

### BACKGROUND

NAD⁺ is an essential component of cellular processes necessary to support various metabolic functions. The classic role of NAD⁺ is a co-enzyme that catalyzes cellular redox reactions, becoming reduced to NADH, in many fundamental metabolic processes, such as glycolysis, fatty acid beta oxidation, or the tricarboxylic acid cycle. NAD+ functions as an electron carrier in energy metabolism of amino acids, fatty acids and carbohydrates (Bogan & Brenner, 2008). In addition, NAD⁺ is critical for redox reactions and as a substrate for signaling by the D′-consuming enzymes, poly-Adenosine-ribose polymerases (PARPs), the sirtuins (SIRT1 to SIRT7), and CD38/157 ectoenzymes, in the regulation of DNA repair, energy metabolism, cell survival and circadian rhythms (Bonkowski, M. S. & Sinclair, D., Nat. Rev. Mole. Cell. Bio., 17, 679-690, 2016)).

NAD⁺ levels decline at cellular, tissue/organ, and organismal levels with age. Activities of NAD-consuming enzymes are affected by this NAD⁺ decline, contributing to a broad range of age-associated pathophysiologies. It has been observed that increasing the level of NAD+ during the aging process improves glucose metabolism and mitochondrial function. Raising NAD⁺ concentrations delays aging in yeast, flies and mice (Mouchiroud et al. Cell 154, 464-471, 2014). In addition, it also has been demonstrated that NAD⁺ directly regulates protein-protein interactions, the modulation of which may protect against cancer and radiation exposure as well as having a direct impact on aging (Li et al., Science 355, 1312-1317, 2017).

There are several precursors and intermediates to synthesize NAD+: tryptophan, nicotinamide, nicotinic acid (NA), nicotinamide riboside (NR), and nicotinamide mononucleotide (NMN), nicotinic acid ribose (NaR) and nicotinic acid mononucleotide (NaMN). These precursors and intermediates and derivatives thereof have a wide range of biological activity and supplementation with NAD precursors and their derivatives have been associated with increased physical endurance, health span and longevity in mammals by increasing NAD+ levels.

Flavonoids and their polymers comprise one of the largest groups of phytonutrients that afford beneficial health effects. These compounds are senolytic agents that selectively target and induce apoptosis/death of senescent cells (Kirkland, et al. 2017 J Am GeriatrSoc 65(10):2297-2301; Zhu, et al. 2015 Aging Cell 14(4):644-658). Cellular senescence, which increases with age, occurs when an accumulation of DNA damage causes a change in the state of the chromatin of proliferating or terminally differentiated non-dividing cells. Subsequently, these cells stop dividing and become resistant to apoptosis. Flavonoid polyphenols, have been shown to have effect on health and lifespan through their senolytic activity.

Flavonoid polyphenols, such as fisetin, morin, quercetin, isorhamnetin, luteolin and myricetin have been investigated and shown to have a number of biological activities which may contribute to their senolytic activity. For example, fisetin (3,3′,4′,7-tetrahydroxyflavone) is a bioactive flavonol present in various human foods and is associated with antiproliferative, apoptotic, antioxidant and neuroprotective activities (Khan et al., Antioxidants & Redox Signaling 19(2):151-162, 2013). Fisetin also has been reported to inhibit human low-density lipoprotein (LDL) oxidation in vitro (Shia et al., Agric. Food Chem 57:83-89, 2009)), induce quinone oxidoreductase activity (Kimura et al., I. Epidemiol. 8:168-175, 1998), and induce neurite outgrowth by activating ERK1/2 and PC12 differentiation (Szliska et al., Int. J. Oncol. 39:771-779, 2011).

Accordingly, despite the myriad of approaches being investigated, there is an ongoing need for the development of formulations which mitigate the physiological decline associated with aging and age-related conditions.

### SUMMARY

Provided herein are formulations comprising an NAD+ agonist, formulations comprising a flavonoid polyphenol, formulations comprising a combination of an NAD+ agonist and a flavonoid polyphenol. Also provided herein are methods of administering a combination of these formulations for use in methods for treating, ameliorating, or preventing (i.e., reducing the likelihood of occurrence of) at least one symptom or indication an age-related disease or disorder in a subject.

The formulations and methods provided herein result in the selective apoptosis and removal of senescent cells (senolysis) induced by the flavonoid polyphenol (e.g., fisetin) and an increase in NAD+ concentrations in healthy cells induced by the NAD+ agonist. Accordingly, use of the formulations provided herein provide a synergistic effect on the treatment of age-related conditions, e.g., cardiovascular and metabolic diseases.

Accordingly, in one aspect, provided herein is combination comprising (a) a first formulation comprising an NAD+ agonist, pharmaceutically acceptable salt, derivative or metabolite thereof; and (b) a second formulation comprising a polyphenol flavonoid. In some embodiments, the NAD+ agonist is the only active agent in the first formulation, and the polyphenol flavonoid is the only active agent in the second formulation. In some embodiments, the NAD+ agonist is the only active agent in the first formulation and the second formulation comprises a flavonoid polyphenol and an NAD+ agonist.

In another aspect, provided herein is a method of ameliorating, treating and/or delaying an age-related disease or disorder in a subject in need thereof, the method comprising administering and effective amount (a) a formulation comprising an NAD+ agonist; and (b) a formulation comprising a polyphenol flavonoid.

In another aspect, provided herein is a method of enhancing or preventing the decline of cognitive performance in a subject in need thereof, the method comprising administering and effective amount (a) a formulation comprising an NAD+ agonist; and (b) a formulation comprising a polyphenol flavonoid.

Also provided herein are kits and articles of manufacture comprising the first and second formulations.

Other features and advantages of the formulations provided herein will be apparent from the following description, the examples, and from the claims. All publications, patent applications, patents, and other references mentioned herein are incorporated by reference in their entirety.

### BRIEF DESCRIPTION OF THE DRAWINGS

FIG. 1 is a graphic depiction of Treated Whole Blood Concentration of NAD+ in Individual Beagle Dogs, Group 1, on day −7 and Following Oral Administration of NAR on Days 1, and 7 (10 mg/kg/day).

FIG. 2 is a graphic depiction of Treated Whole Blood Concentration of NAD+ in Individual Beagle Dogs, Group 2, on day −7 and Following Oral Administration of NAR on Days 1, and 7 (20 mg/kg/day).

FIG. 3 is a graphic depiction of Treated Whole Blood Concentration of NAD+ in Individual Beagle Dogs, Group 3, on day −7 and Following Oral Administration of NAR on Days 1, and 7 (30 mg/kg/day).

FIG. 4 is a graphic depiction of Cmax, Treated Whole Blood Concentration of NAD+ in Individual Beagle Dogs, Group 1-3, on day −7 and Following Oral Administration of NAR on Days 1, and 7.

FIG. 5 is a graphic depiction of AUC₀₋₂₄, Treated Whole Blood Concentration of NAD+ in Individual Beagle Dogs, Group 1-3, on day −7 and Following Oral Administration of NAR on Days 1, and 7.

FIG. 6 is a graphic depiction of Treated Whole Blood Ratio of NAD+ in Individual Beagle Dogs Following Oral Administration of NAR on Days 1 and 7.

FIGS. 7A-7C are graphic depictions of the Frailty Index (FIG. 7A), FRIGHT Age (FIG. 7B) and AFRAID score (FIG. 7C) of mice treated with low and high doses of NaR (60 and 400 mg/kg, respectively). Samples were taken monthly from 18-30 months of age.

FIGS. 8A and 8B are graphic depictions of the whole blood NAD concentration and overall exposure (AUC₀₋₂₄) relative to NaR and fisetin dose levels in beagles from Example 6, Study #1. FIG. 8A is a plot showing the whole blood NAD concentration relative to overall exposure (AUC₀₋₂₄) of differing dose levels of NaR and fisetin over a period of 24 hours. The square cells have the same scale, with the upper right-hand quadrant of each square being the desired outcome (a high concentration of NAD and a high ACU₀₋₂₄ value).

FIG. 8B is a plot of the whole blood concentration of NAD in beagles administered 20 mg/kg NaR and 40 mg/kg fisetin. Following a peak concentration on Day 2, NAD levels then decrease to baseline on days 3, 4, 5 and 7 despite daily dosing with NaR. Without further administration of NaR or fisetin, the NAD levels then increased on days 21 and 35.

FIGS. 9A and 9B are graphic depictions of the whole blood NAD concentrations, and overall exposure (AUC₀₋₁₂) relative to NaR and fisetin dose levels from Example 6, Study #2. FIG. 9A a plot showing the whole blood NAD concentration relative to overall exposure (AUC₀₋₁₂) obtained from a dose regimen of NaR 20 mg/kg and fisetin 20 mg/kg over a period of 12 hours. FIG. 9B is a plot of the whole blood concentration of NAD obtained from the administration of 20 mg/kg NaR and 20 mg/kg fisetin. The NAD concentrations over the course of 12 hours on days 14, 21 and 28 do not recover as compared with previous days.

FIGS. 10A-10E are graphical depictions of the whole blood NAD concentrations (ng/mL) and overall exposure (AUC₀₋₁₂, and AUC₀₋₂₄) correlation analysis from Example 6, Studies #1 and #2. FIGS. 10A and 10B are plots depicting NAD concentrations and overall exposure over a period of 12 hours and 24 hours, respectively, from the administration of only NaR at varying dose levels. FIG. 10C is a plot depicting the NAD concentration and overall exposure over a period of 12 hours from the administration of only fisetin at a dose of 20 mg/kg. FIGS. 10D and 10E are plots depicting the NAD concentrations and overall exposure over a period of 12 hours and 24 hours, respectively, from the administration of a combination of NaR and fisetin at differing dose levels.

FIGS. 11A-11F are graphical depictions comparing the concentration of NAD (ng/mL) by day for all dosing regimen in Example 6, Studies #1 and #2. The dosage of only NaR (FIGS. 11A-11B) had a median Cₘₐₓ of 6990 ng/mL and a median AUC₀₋₂₄ of 133,000 ng/mL. The dosage of only fisetin (FIGS. 11C-11D) had a median Cₘₐₓ of 5090 ng/mL and a median AUC₀₋₂₄ of 49,900 ng/mL. The combined dosage of NaR with fisetin (FIGS. 11E-11F) had a median Cₘₐₓ of 6290 ng/mL and a median AUC₀₋₂₄ of 125,000 ng/mL.

FIG. 12 is a plot of the whole blood NAD concentration comparing the dosing of 20 mg/kg NaR and 20 mg/kg fisetin vs. 20 mg/kg NAR and 40 mg/kg fisetin from Example 6, Study #2. NaR was administered over 28 days and fisetin administered only on days 1 and 2. Both dosing regimen resulted in a decrease in NAD concentration at days 7 and 14, however, the NAD concentration recovered to above baseline levels at days 28 and 35 in the 20 mg/kg NaR and 40 mg/kg fisetin dosing regimen.

### DETAILED DESCRIPTION

Provided herein are formulations comprising a combination of a nicotinamide (NAD) derivative and a flavonoid polyphenol for use methods of treating, preventing or ameliorating a disease or disorder associated with aging, as well as kits and articles of manufacture, as well as methods for preparation thereof.

While in no way intended to be limiting, suitable applications in which formulations, kits and articles of manufacture provided herein can be used are set forth in this section and exemplified in the working Examples.

### I. Definitions

In order that the present description may be more readily understood, certain terms are first defined. Additional definitions are set forth throughout the detailed description. Unless defined otherwise, all technical and scientific terms used herein have the same meaning as commonly understood by one of ordinary skill in the art, and conventional methods of immunology, protein chemistry, biochemistry, recombinant DNA techniques and pharmacology are employed.

As used herein, the singular forms “a”, “an” and “the” include plural referents unless the context clearly dictates otherwise. The use of “or” or “and” means “and/or” unless stated otherwise. Furthermore, use of the term “including” as well as other forms, such as “include”, “includes”, and “included”, is not limiting.

The term “about” as used herein when referring to a measurable value such as an amount, a temporal duration and the like, is encompasses variations of up to ±10% from the specified value. Unless otherwise indicated, all numbers expressing quantities of ingredients, properties such as molecular weight, reaction conditions, etc., used herein are to be understood as being modified by the term “about”.

As used herein, an “NAD agonist” is an agent which raises NAD+ levels in a cell or blood sample, and/or increases the ratio of NAD+ to NADH levels in a cell or blood sample, and/or increases NAD+ production in a cell or blood sample. Methods for determining the amount of NAD+ in a cell or blood sample, the ratio of NAD+ to NADH in a cell or blood sample, and the production of NAD+ in a cell or blood sample, are known in the art and are described in, for example, Schwartz et al. (1974) J. Biol. Chem. 249:4138-4143; Sauve and Schramm (2003) Biochemistry 42(31):9249-9256; Yamada et al. (2006) Analytical Biochemistry 352:282-285, or can be determined using commercially available kits such as, for example, NAD/NADH-Glo Assay (Promega Inc.) or NAD/NADH Quantitation Colorimetric Kit (BioVision Inc.).

The term “formulation” refers to preparations which are in such form as to permit the biological activity of the active ingredients to be effective, and which contain no additional components which are significantly toxic to the subjects to which the formulation would be administered.

An “aseptic” formulation refers to preparations which are free or essentially free from pathogenic organisms.

The terms “sterile” or “commercially sterile” formulation is aseptic or free or essentially free from all living microorganisms and their spores which can change or degrade the active ingredient.

A “stable” formulation, as used herein, is one in which the active ingredient therein essentially retains its physical stability and/or chemical stability and/or biological activity upon storage.

The terms “carrier”, “nutraceutically acceptable carrier” and “pharmaceutically acceptable carrier” as used herein, encompass carriers, excipients, and diluents that are compatible with other ingredients in the formulations provided herein which are not deleterious to the subject, including materials, composition or vehicles, such as a liquid or solid filler, diluent, excipient, solvent or encapsulating material, involved in carrying or transporting an active agent within the body of a subject.

The term “surfactant” refers to organic substances having amphipathic structures (i.e., composed of groups of opposing solubility tendencies, typically an oil-soluble hydrocarbon chain and a water-soluble ionic group), which can lower the surface tension (or interfacial tension) between two liquids or between a liquid and a solid. Surfactants may act as detergents, wetting agents, emulsifiers, foaming agents, and dispersants. Surfactants can be classified, depending on the charge of the surface-active moiety, into anionic, cationic and non-ionic agents for various pharmaceutical compositions and preparations of biological materials.

A “preservative” is a compound which can be optionally included in the formulation to essentially reduce bacterial action therein, thus facilitating the production of a multi-use formulation, for example. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol.

The terms “treat,” “treating,” and “treatment,” and methods of “treatment” as used herein employ administration to a subject the combination disclosed herein in order to cure, delay, reduce the severity of, or ameliorate one or more symptoms of a disease or disorder or recurring disease or disorder, or in order to prolong the survival of a subject beyond that expected in the absence of such treatment.

The term “effective amount” as used herein refers to an amount of the combination provided herein to alleviate or ameliorate symptoms of disease or to prolong the survival of the subject being treated. Determination of an effective amount is within the capability of those skilled in the art, especially in light of the detailed disclosure provided herein. Effective dosages may be determined by using in vitro and in vivo methods.

As used herein, “administering” refers to the physical introduction of a composition comprising the formulations provided herein to a subject, using any of the various methods and delivery systems known to those skilled in the art. Preferred routes of administration for formulations described herein include oral, topical, epidermal or mucosal route of administration, for example, intranasally, orally, vaginally, rectally, sublingually. Other routs of administration include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intra-arterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Administering can also be performed, for example, once, a plurality of times, and/or over one or more extended periods.

The terms “prophylactic use” or “prophylaxis” used herein refer to decreasing the likelihood of, or prevention of, a disease or condition (e.g., an age-related disease or condition).

“Optimal biologic dose (OBD)” is defined as the minimum dose of a compound that gives the most optimal and lasting in vivo response without clinically unacceptable toxicity.

The term “bioavailable” when referring to a compound is art-recognized and refers a form of a compound that allows for it, or a portion of the amount of compound administered, to be absorbed by, incorporated to, or otherwise physiologically available to a subject or patient to whom it is administered.

As used herein, the term “chronically” (e.g., to chronically administer a compound), or similar terms, refers to a method of administration in which the formulations provided herein are administered to a subject in an amount and with a frequency sufficient to maintain an effective amount of the agent in the subject for at least one (e.g., at least two, three, four, five, or six) month(s). In some embodiments, a formulation provided herein can be chronically administered to a subject for a year or more.

The term “disorder” is used in this disclosure to mean, and is used interchangeably with, the terms disease, condition, or illness, unless otherwise indicated.

As used herein, the term “subject” includes any human or non-human animal. The term “non-human animal” includes all vertebrates, e.g., mammals and non-mammals, such as mouse, rat, guinea pig, dog, cat, horse, cow, pig, or non-human primate, such as a monkey, chimpanzee, baboon or rhesus.

The term “sample” refers to a collection of fluids, cells or tissues isolated from a subject. Biological fluids are typically liquids at physiological temperatures and may include naturally occurring fluids present in, withdrawn from, expressed or otherwise extracted from a subject or biological source. Examples of biological fluids include blood, serum, serosal fluids, plasma, lymph, urine, cerebrospinal fluid, saliva, ocular fluids, cystic fluid, tear drops, feces, sputum, mucosal secretions, vaginal secretions, gynecological fluids, ascites fluids, fluids of the pleural, pericardial, peritoneal, abdominal and other body cavities, fluids collected by bronchial lavage and the like.

The term “control sample”, as used herein, refers to any clinically relevant control sample, including, for example, a sample from a healthy subject or a sample made at an earlier timepoint from the subject to be assessed.

Various aspects described herein are described in further detail in the following subsections.

### II. NAD Agonists and Derivatives

Suitable NAD+ agonists which can be used in the first formulation provided herein include NAD+ precursors and derivatives thereof, including but not limited to one or a combination of two or more of nicotinamide mononucleotide (NMN), nicotinamide riboside (NR), nicotinic acid riboside (NaR), ester derivatives of nicotinic acid riboside, nicotinic acid (niacin), ester derivatives of nicotinic acid, nicotinic acid mononucleotide (NaMN), ester derivatives of nicotinic acid mononucleotide, nicotinic acid adenine dinucleotide (NaAD), nicotinic acid adenine dinucleotide (NAAD), or a pharmaceutically acceptable salts, derivatives or prodrugs thereof.

NAD+ precursors and derivatives thereof are known in the art and can be produced using commercially available starting materials or synthesized using known organic, inorganic and/or enzymatic processes, for example, as described in U.S. Pat. No. 8,106,184, U.S. patent Ser. No. 10/654,883, U.S. patent Ser. No. 10/183,036, WO 2019/221813, WO 2019/222368, WO 2020/028682, WO 2020/028684, WO 2019/222360, and WO 2019/023748, WO 2019/226755, the contents of each of which are incorporated by reference.

In some embodiments, the first formulation comprises NaR or a pharmaceutically acceptable salt, derivative or prodrug thereof. In some embodiments, the formulation comprises a salt of NaR.

In some embodiments, first formulation comprises an inorganic salt of NaR. In some embodiments, the inorganic salt of NaR comprises Li+, Na+, K+, Rb+, Cs+, Be2+, Mg2+, Ca2+, Sr2+, Zn2+ and Ba2+ or a combination thereof. In one embodiment, the inorganic salt of NaR comprises Na+. In one embodiment, the inorganic salt of NaR comprises K+.

In some embodiments, the first formulation comprises an amino acid salt of NaR. The amino acid salt of NaR can comprise any one of the twenty naturally occurring amino acids or derivatives thereof. In some embodiments, the NaR salt is a mono-amino acid salt. In some embodiments, the NaR salt is a di-amino acid salt. In some embodiments, the amino acid salt of NaR is selected from phenylalanine, isoleucine, tryptophan, lysine, asparagine, valine, aspartic acid, alanine, arginine and proline.

In some embodiments, the amino acid salt of NaR is selected from
(S)-2-Ammonio-3-phenylpropanoate compound with 1-((2R,3R,4S,5R)-3,4-di hydroxy-5-(hydroxymethyl)tetrahydrofuran-2-yl)pyridine-1-ium-3-carboxylate (1:1);
(2S,3S)-2-Ammonio-3-methylpentanoate compound with 1-((2R,3R,4S,5R)-3,4-dihydroxy-5-(hydroxymethyl)tetrahydrofuran-2-yl)pyridine-1-ium-3-carboxylate (1:1);
(S)-2-Ammonio-3-(1H-indol-3-yl)propanoate compound with 1-((2R,3R,4S,5R)-3,4-dihydroxy-5-(hydroxymethyl)tetrahydrofuran-2-yl)pyridine-1-ium-3-carboxylate (1:1);
1-((2R,3R,4 S,5R)-3,4-dihydroxy-5-(hydroxymethyl)tetrahydrofuran-2-yl)pyridin-1-ium-3-carboxylate-(S)-6-amino-2-ammoniohexanoate (1:1);
1-((2R,3R,4S,5R)-3,4-dihydroxy-5-(hydroxymethyl)tetrahydrofuran-2-yl)pyridin-1-ium-3-carboxylate (S)-5-amino-2-ammonio-5-oxopentanoate (1:1);
1-((2R,3R,4S,5R)-3,4-dihydroxy-5-(hydroxymethyl)tetrahydrofuran-2-yl-pyridin-1-ium-3-carboxylate-(S)-2-ammonio-4-methylpentanoate (1:1);
1-((2R,3R,4S,5R)-3,4-dihydroxy-5-(hydroxymethyl)tetrahydrofuran-2-yl)pyridin-1-ium-3-carboxylate-(S)-2-ammonio-4-carboxybutanoate (1:1);
(S)-2-ammonio-3-methylbutanoate-1-((2R,3R,4S,5R)-3,4-dihydroxy-5-(hydroxymethyl)tetrahydrofuran-2-yl)pyridin-1-ium-3-carboxylate (1:1);
(S)-2-ammonio-5-guanidinopentanoate-1-((2R,3R,4S,5R)-3,4-dihydroxy-5-(hydroxymethyl)tetrahydrofuran-2-yl)pyridin-1-ium-3-carboxylate (1:1); and
1-((2R,3R,4S,5R)-3,4-dihydroxy-5-(hydroxymethyl)tetrahydrofuran-2-yl)pyridin-1-ium-3-carboxylate-(S)-2-ammonio-3-(1H-imidazol-4-yl)propanoate (1:1).

In some embodiments, the amino acid salt of NaR is (2S,3S)-2-Ammonio-3-methylpentanoate compound with 1-((2R,3R,4S,5R)-3,4-dihydroxy-5-(hydroxymethyl)tetrahydrofuran-2-yl)pyridine-1-ium-3-carboxylate (1:1); or (S)-2-Ammonio-3-(1H-indol-3-yl)propanoate compound with 1-((2R,3R,4S,5R)-3,4-dihydroxy-5-(hydroxymethyl)tetrahydrofuran-2-yl)pyridine-1-ium-3-carboxylate (1:1).

In one embodiment, the amino acid salt of NaR is the tryptophan salt.

In some embodiments, the first formulation comprises NaMN, or a pharmaceutically acceptable salt, derivative or prodrug thereof. In some embodiments, the first formulation comprises a salt of NaMN.

In some embodiments, first formulation comprises an inorganic salt of NaMN. In some embodiments, the inorganic salt of NaMN comprises Li+, Na+, K+, Rb+, Cs+, Be2+, Mg2+, Ca2+, Sr2+, Zn2+ and Ba2+ or a combination thereof. In one embodiment, the inorganic salt of NaMN comprises Na+. In one embodiment, the inorganic salt of NaR comprises K+. In one embodiment, the inorganic salt of NaR comprises Na+ and K+.

In some embodiments, the first formulation comprises an inorganic salt of NaMN selected from:
Sodium 1-((2R,3R,4S,5R)-5-(((hydrogenphosphonato)oxy)methyl)-3,4-dihydroxytetrahydrofuran-2-yl)pyridine-1-ium-3-carboxylate;
Potassium 1-((2R,3R,4S,5R)-5-(((hydrogenphosphonato)oxy)methyl)-3,4-dihydroxytetrahydraofuran-2-yl pyridine-1-ium-3-carboxylate:
Sodium 1-((2R,3R,4S,5R)-3,4-dihydroxy-5-((phosphonatooxy)methyl)-tetrahydrofuran-2-yl)pyridine-1-ium-3-carboxylate;
Potassium 1-((2R,3R,4S,5R)-3,4-dihydroxy-5-((phosphonatooxy)methyl)-tetrahydrofuran-2-yl)pyridine-1-ium-3-carboxylate;
Potassium sodium 1-((2R,3R,4S,5R)-3,4-dihydroxy-5-((phosphonatooxy)methyl)tetrahydrofuraran-2-yl)pyridine-1-ium-3-carboxylate;
Sodium 1-((2R,3R,4R,5R)-3,4-diacetoxy-5-(((hydrogenphosphonato)oxy)methyl)tetrahydrofuran-2-yl)pyridine-1-ium-3-carboxylate;
Potassium 1-((2R,3R,4R,5R)-3,4-diacetoxy-5-(((hydrogenphosphonato)oxy)methyl)tetrahydrofuran-2-yl)pyridine-1-ium-3-carboxylate;
Lithium 1-((2R,3R,4S,5R)-5-(((hydrogenphosphonato)oxy)methyl)-3,4-dihydroxytetrahydrofuran-2-yl)pyridin-1-ium-3-carboxylate;
Lithium 1-((2R,3R,4S,5R)-2,4-dihydroxy-5-((phosphonato)oxy)methyl)tetrahydrofuran-2-yl)pyridin-1-ium-3-carboxylate;
Calcium 1-((2R,3R,4S,5R)-5-(((hydrogenphosphonato)oxy)methyl)-3,4-dihydroxytetrahydrofuran-2-yl)pyridin-1-ium-3-carboxylate; and
Magnesium 1-((2R,3R,4S,5R)-5-(((hydrogenphosphonato)oxy)methyl)-3,4-dihydroxytetrahydrofuran-2-yl)pyridine 1-ium-3-carboxylate;
   or a combination thereof.

In certain embodiments, the first formulation comprises sodium 1-((2R,3R,4S,5R)-5-(((hydrogenphosphonato)oxy)methyl)-3,4-dihydroxytetrahydrofuran-2-yl)pyridine-1-ium-3-carboxylate; potassium 14(2R,3R,4S,5R)-3,4-dihydroxy-5-((phosphonatooxy)methyl)-tetrahydrofuran-2-yl)pyridine-1-ium-3-carboxylate; potassium sodium 1-((2R,3R,4S,5R)-3,4-dihydroxy-5-((phosphonatooxy)methyl)tetrahydrofuraran-2-yl)pyridine-1-ium-3-carboxylate; Potassium 1-((2R,3R,4R,5R)-3,4-diacetoxy-5-(((hydrogenphosphonato)oxy)methyl)tetrahydrofuran-2-yl)pyridine-1-ium-3-carboxylate, or a combination thereof.

In some embodiments, the first formulation comprises an amino acid salt of NaMN. The amino acid salt of NaMN can comprise any one of the twenty naturally occurring amino acids or derivatives thereof. In some embodiments, the NaMN salt is a mono-amino acid salt. In some embodiments, the NaMN salt is a di-amino acid salt. In some embodiments, the amino acid salt of NaMN is selected valine, glutamine, histidine, arginine, tryptophan, threonine, proline, lysine, methionine, leucine, isoleucine, and valine.

In some embodiments, the amino acid salt of NaMN is selected from the mono L-valine salt, the mono L-glutamine salt, the di L-valine salt, the di L-glutamine salt, the mono L-histidine salt, the di L-histidine salt, the mono L-arginine salt, the mono L-tryptophan salt, the di L-arginine salt, the di L-glutamic salt, the di L-tryptophan salt, the di L-threonine salt, the di L-lysine salt, the mono L-methionine salt, the di L-leucine salt and the di D-valine salt.

In some embodiments, the first formulation comprises an amino acid salt of NaMN selected from:
(S)-1-Carboxy-2-methylpropan-1-aminium 1-(2R,3R, 4S, 5R)-5-(((hydrogenphosphonato)oxy)methyl)-3,4-dihydrohytetrahydrofuran-2-yl)pyridine-1-ium-3-carboxylate;
(S)-4-Amino-1-carboxy-4-oxobutan-1-aminium 1-((2R,3R, 4S, 5R)-5-(((hydrogen phosphonato)oxy)methyl)-3,4-dihydrohytetrahydrofuran-2-yl)pyridine-1-ium-3-carboxylate;
Bis((S)-1-Carboxy-2-methyl propan-1-aminium)-1-((2R,3R,4S,5R)-3,4-dihydroxy-5-((phosphonatooxy)methyl)tetrahydrofuran-2-yl)pyridin-1-ium-3-carboxylate;
Bis((S)-4-Amino-1-carboxy-4-oxobutan-1-aminium)-1-((2R,3R,4S,5R)-3.4-dihydroxy-5-((phosphonatooxy)methyl)tetrahydrofuran-2-yl)pyridin-1-ium-3-carboxylate;
(S)-1-Carboxy-2-(1H-imidazol-4-yl)ethanaminium 1-((2R,3R,4S,5R)-5-(((hydrogenphosphonato)oxy)methyl)-3.4-dihydroxytetrahydroguran-2-yl)pyridin-1-ium-3-carboxylate;
Bis((S)-1-Carboxy-2-(1H-imidazol-411)ethanaminium)-1-((2R,3R,4S,5R)-3,4-dihydroxy-5-((phosphonatooxy)methyl)tetrahydrofuran-2-yl)pyridin-1-ium-3-carboxylate;
(S)-1-Carboxy-4-guanidinobutan-1-aminium 1-((2R,3R,4S,5R)-5-(((hydrogenphosphonato)oxy)methyl-3,4-d hydroxytetrahydrofuran-2-yl)pyridin-1-ium-3-carboxylate;
(S)-1-Carboxy-2-(1H-indol-3-yl)ethanaminium 1-((2R,3R4S,5R)-5-(((hydrogenphosphonato)oxy)methyl)-3,4-dihydroxytetrahydrofuran-2-yl)pyridin-1-ium-3-carboxylate;
Bis((S)-1-Carboxy-4-guanidinbutan-1-aminium)-1-((2R,3R,4S,5R)-3,4-dihydroxy-5-((phosphonatooxy)methyl)tetrahydrofuran-2-yl)pyridin-1-ium-3-carboxylate;
Bis((S)-1,3-Dicarboxypropan-1-aminium)-1-(2R,3R,4S,5R)-3,4-dihydroxy-5-((phosphonatooxy)methyl)tetrahydrofuran-2-yl)pyridin-1-ium-3-carboxylate;
Bis((S)-1-Carboxy-2-(1H-indol-3-yl)ethanaminium)-1-((2R,3R,4S,5R)-3,4-dihydroxy-5-((phosphonatooxy) methyl)tetrahydrofuran-2-yl)pyridin-1-ium-3-carboxylate;
Bis((1S,2R)-1-carboxy-2-hydroxypropan-1-aminium)-1-((2R,3R,4S,5R)-3,4-dihydroxy-5-((phosphonatooxy)methyl)tetrahydrofuran-2-yl)pyridin-1-ium-3-carboxylate;
(S)-5-amino-5-carboxypentan-1-aminium 1-((2R,3R,4S,5R)-3,4-dihydroxy-5-((phosphonatooxy)methyl)tetrahydrohuran-2-yl)pyridine-1-ium-3-carboxylate;
(S)-1-carboxy-3-(methylthio)propan-1-aminium 1-((2R,3R,4S,5R)-5-(((hydrogenphosphonato)oxy)methyl)-3,4-dihydroxytetrahydrofuran-2-yl)pyridin-1-ium-3-carboxylate;
(S)-1-Carboxy-3-methylbutan-1-aminium 1-((2R,3R,4S,5R)-3,4-dihydroxy-5-((phosphonatooxy)methyl)tetrahydrofuran-2-yl)pyridin-1-ium-3-carboxylate, or a combination thereof.

In some embodiments, the first formulation comprises NMN, or a pharmaceutically acceptable salt, derivative or prodrug thereof. In some embodiments, the first formulation comprises a salt of NMN.

In some embodiments, first formulation comprises an inorganic salt of NMN. In some embodiments, the inorganic salt of NMN comprises Li+, Na+, K+, Rb+, Cs+, Be2+, Mg2+, Ca2+, Sr2+, Zn2+ and Ba2+ or a combination thereof.

In some embodiments, the inorganic salt of NMN is selected from the following:
Sodium ((2R,3S,4R,5R)-5-(3-carbamoylpyridin-1-ium-1-yl)-3,4-dihydroxytetrahydrofuran-2-yl)methyl phosphate;
Potassium ((2R,3S,4R, 5R)-5-(3-carbamoylpyridin-1-ium-1-yl)-3,4-dihydroxytetrahydrofuran-2-yl)methylphosphate;
Calcium((2R,3S,4R,5R)-5-(3-carbamoylpyridin-1-ium-1-yl)-3,4-dihydroxytetrahydrofuran-2-yl)methyl phosphate;
Magnesium ((2R,3S,4R,5R)-5-(3-carbamoylpyridin-1-ium-1-yl)-3,4-dihydroxytetrahydrofuran-2-yl) methyl phosphate;
Lithium ((2R,3S,4R,5R)-5-(3-carbamoylpyridin-1-ium-1-yl)-3,4-dihydroxytetrahydrofuran-2-yl)methyl phosphate;
   or a combination thereof.

In some embodiments, the first formulation comprises am amino acid salt of NMN. The amino acid salt of NMN can comprise any one of the twenty naturally occurring amino acids or derivatives thereof. In some embodiments, the NMN salt is a mono-amino acid salt. In some embodiments, the NMN salt is a di-amino acid salt. In some embodiments, the amino acid salt of NMN is a mono L-valine salt, a mono L-tryptophan salt, a mono L-proline salt, or combination thereof.

In some embodiments, the first formulation comprises an amino acid salt of NMN selected from:
(S)-1-Carboxy-2-methylpropan-1-aminium ((2R,3S, 4R, 5R)-5-(3-carbamoylpyridin-1-ium-1-yl)-3,4-dihydroxytetrahydrofuran-2-yl)methyl phosphate;
S)-1-Carboxy-2-(1H-indol-3-yl)ethanaminium ((2R,3S,4R,5R)-5-(3-carbamoylpyridin-1-ium-1-yl)-3,4-dihydroxytetrahydrofuran-2-yl)methyl phosphate;
S)-2-carboxypyrrolidin-1-ium ((2R,3S,4R,5R)-5-(3-carbamoylpyridin-1-ium-1-yl)-3,4-dihydroxytetrahydrofuran-2-yl)methyl phosphate;
   or a combination thereof.

In some embodiments, the first formulation comprises nicotinic acid (NA) or a pharmaceutically acceptable salt, derivative or prodrug thereof. In some embodiments, the first formulation comprises a salt of NA. In some embodiments, the first formulation comprises an inorganic salt of NA, e.g., a Li+, Na+, K+, Rb+, Cs+, Be2+, Mg2+, Ca2+, Sr2+, Zn2+, Ba2+ salt or a combination thereof.

In some embodiments, the first formulation comprises an amino acid salt of NA.

In some embodiments, the first formulation comprises an amino acid salt of NA selected from:
Nicotinic acid L-arginine salt;
Nicotinic acid L-glutamic acid salt;
(S)-1-carboxy-2-(1H-imadazol-4-yl)ethanaminium nicotinate;
(S)-2-carboxypyrrolidin-1-ium nicotinate;
(S)-1-carboxy-2-methylpropan-1-aminium nicotinate;
(S)-1-carboxy-2-(1H-indol-3-yl)ethanaminium nicotinate;
2-hydroxy-N,N,N-trimethylanaminiumn nicotinate;
(R)-3-carboxy-2-hydroxy-N,N,N-trymethylpropan-1-aminium nicotinate;
(S)-1-carboxy-3-(methylthio)propan-1-aminium nicotinate;
(1S,2S)-1-carboxy-2-methylbutan-1-aminium nicotinate;
(R)-1-carboxy-methylpropan-1-aminium nicotinate;
(1S,2S)-1-carboxy-methylbutan-1-aminium nicotinate;
(S)-1-carboxy-methylbutan-1-aminium nicotinate;
Nicotinic acid with (R)-5-((S)-1,2-dihyroxyethyl)-3,4-dihydroxyfuran-2(5H)-one (1:1);
   or a combination thereof.

### III. Polyphenol Flavonoids

Suitable polyphenol flavonoids for use in the second formulation provided herein include fisetin; quercetin; rutin; quercitrin; catechin; gallocatechin; catechin 3-gallate; gallocatechin 3-gallate; epicatechin; epigallocatechin; epicatechin 3-gallate; epigallocatechin3-gallate; theaflavin-3-gallate; theaflavin-3′-gallate; theaflavin-3,3′-digallate; kaempferol; myricetin; galangin; isorhamnetin; pachypodol; rhamnazin; a pyranoflavonol; furanoflavonol; luteolin; resveratrol; apigenin; tangeritin; hesperetin; naringenin; eriodictyol; homoeriodictyol; metabolites, analogs and derivatives thereof; as well as a combination of two or more thereof. In certain embodiments, the second formulation comprises fisetin or a metabolite (e.g., geraldol), analog or derivative thereof.

Flavonoids, analogs, metabolites and derivatives thereof are known in the art and commercially available (e.g., Biomol, Sigma/Aldrich, Indofine). Alternatively, they can be produced using commercially available starting materials using known organic, inorganic and/or enzymatic processes, for example, as described in AU 201112012 and U.S. Pat. No. 9,241,916.

In certain embodiments, the flavonoid is fisetin (3,3′,4′,7-tetrahydroxyflavone) or an analog, derivative or metabolite thereof. In some embodiments, the flavonoid is a glucuronidated, sulfated or methylated metabolite of fisetin. In some embodiments, the flavonoid is glucuronidated fisetin. In some embodiments, the flavonoid is a metabolite of fisetin, e.g., geraldol (3,4′,7-trihydroxy-3′-methoxyflavone). In some embodiments, the flavonoid is glucuronidated geraldol.

### IV. Formulations

Depending on the intended mode of administration, the formulations provided herein can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can also be administered in intravenous (both bolus and infusion), intraperitoneal, subcutaneous or intramuscular form, and all using forms well known to those skilled in the pharmaceutical arts.

One or more other pharmaceutically acceptable carriers, excipients or stabilizers such as those described in Remington's Pharmaceutical Sciences 16th edition, Osol, A. Ed. (1980) also may be included in the formulations provided that they do not significantly adversely affect the desired characteristics of the formulation. Acceptable carriers, excipients or stabilizers are nontoxic to recipients at the dosages and concentrations employed and include: additional buffering agents; co-solvents; antioxidants; chelating agents such as EDTA; and/or biodegradable polymers such as polyesters.

For example, in some embodiments, the first and second formulations provided herein are in the form of tablets, capsules or tablets. Suitable carriers include, without limitation, purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; lubricants, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; a binders, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, algic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the salt such as cyclodextrin, hydroxypropyl-cyclodextrin, PEG400, PEG200.

In certain embodiments, the first and second formulations are suitable for oral administration, such as solid forms including capsules, tablets, pills, granules, or in a liquid form including solutions, suspensions or emulsion.

In certain embodiments, the one or both formulations are in tablet form containing the active component in admixture with non-toxic orally acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets may be uncoated or coated by known techniques to delay disintegration and absorption in the gastrointestinal tract and thereby provide a sustained action over a longer period. For example, a time delay material such as glyceryl monostearate or glyceryl stearate can be employed.

In certain embodiments, one or both formulations are in the form of a capsule containing the active ingredient mixed with an inert solid diluent, for example, calcium carbonate, calcium phosphate or kaolin, or as soft gelatin capsules wherein the composition comprising the active components is mixed with water or an oil medium, for example, peanut oil, liquid paraffin or olive oil.

The formulations provided herein can be prepared according to conventional mixing, granulating or coating methods, respectively. For example, carriers included in the formulations provided herein can include can be either solid or liquid. Solid form preparations include powders, tablets, dispersible granules, capsules, cachets and suppositories. The powders and tablets may be comprised of from about 5 to 45% by weight of the active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pa.

The formulations of the invention may be sterilized by various sterilization methods, including sterile filtration, radiation, etc. In one embodiment, the formulation is filter-sterilized with a presterilized 0.22-micron filter. Sterile compositions can be formulated according to conventional pharmaceutical practice as described in “Remington: The Science & Practice of Pharmacy”, 21st ed., Lippincott Williams & Wilkins, (2005).

### A. First Formulation

In certain embodiments, the first formulation comprising an NAD+ agonist provided herein is formulated for oral administration. In preferred embodiments, the first formulation is in the form of a solid including but not limited to capsules, tablets or pills.

Composition intended for oral use can be prepared according to any method known in the art for the manufacture of effective compositions; and such compositions can contain one or more additional agents as disclosed herein. For example, in some embodiments, formulations in the form of tablets can be film coated or enteric coated according to methods known in the art.

The concentrations of the NAD+ agonist (e.g., NaR, NMN, NaMN and salts thereof) in the first formulation is determined, according to the desired dose and mode of administration. In some embodiments, the NAD+ agonist is formulated to deliver at least about 2 mg/kg of the NAD+ agonist to the subject. In some embodiments, the NAD+ agonist is formulated to deliver between about 2 mg/kg and 100 mg/kg, about 5 mg/kg to 100 mg/kg, 10 mg/kg to 100 mg/kg, about 5 mg/kg, 10 mg/kg, 15 mg/kg, 20 mg/kg, 25 mg/kg, 30 mg/kg, 40 mg/kg, 50 mg/kg, 60 mg/kg, 70 mg/kg, 75 mg/kg, 80 mg/kg, 90 mg/kg or about 100 mg/kg. Ranges intermediate to the above recited concentrations can also be used. For example, ranges of values using a combination of any of the above recited values as upper and/or lower limits are intended to be included.

Accordingly, the concentration of the NAD+ agonist in the first formulation is between about 0.5 mg to about 10,000 mg. In some embodiments, the first formulation comprises at least 2 5, 20, 50, 75, 100, 150, 250, 500, 750, 1000, 1250, 2500, 3500, 4000, 5000, 6000 mg or 7000 mg of the NAD+ agonist, or in a range of from one amount to another amount in the list of doses.

### B. Second Formulation

In certain embodiments, the second formulation comprising a flavonoid. In certain embodiments, the second formulation further comprises an NAD+ agonist. In some embodiments, the second formulation is formulated for oral administration. In preferred embodiments, the second formulation is in the form of a solid including but not limited to capsules, tablets or pills.

In certain embodiments, the second formulation comprises fisetin, quercetin; rutin; quercitrin; catechin; gallocatechin; catechin 3-gallate; gallocatechin 3-gallate; epicatechin; epigallocatechin; epicatechin 3-gallate; epigallocatechin3-gallate; theaflavin-3-gallate; theaflavin-3′-gallate; theaflavin-3,3′-digallate; kaempferol; myricetin; galangin; isorhamnetin; pachypodol; rhamnazin; a pyranoflavonol; furanoflavonol; luteolin; resveratrol; apigenin; tangeritin; hesperetin; naringenin; eriodictyol; homoeriodictyol; analogs and derivatives thereof; as well as a combination of two or more thereof.

In certain embodiments, the second formulation comprises fisetin (3,3′,4′,7-tetrahydroxyflavone) or an analog, derivative or metabolite thereof. In some embodiments, the second formulation comprises a glucuronidated, sulfated or methylated metabolite of fisetin. In some embodiments, the second formulation comprises glucuronidated fisetin. In some embodiments, the second formulation comprises geraldol (3,4′,7-trihydroxy-3′-methoxyflavone). In some embodiments, the second formulation comprises glucuronidated geraldol.

The concentrations of the flavonoid the second formulation is determined, according to the desired dose and mode of administration. In some embodiments, the flavonoid formulation is formulated to deliver at least about 10 mg/kg of the flavonoid to the subject. In some embodiments, the flavonoid is formulated to deliver between about 10 mg/kg and 500 mg/kg, about 10 mg/kg to 200 mg/kg, about 10 mg/kg to 100 mg/kg, about 10 mg/kg, 15 mg/kg, 20 mg/kg, 25 mg/kg, 30 mg/kg, 40 mg/kg, 50 mg/kg, 60 mg/kg, 70 mg/kg, 75 mg/kg, 80 mg/kg, 90 mg/kg or about 100 mg/kg. Ranges intermediate to the above recited concentrations can also be used. For example, ranges of values using a combination of any of the above recited values as upper and/or lower limits are intended to be included.

Accordingly, the concentration of the flavonoid in the second formulation is between about 1 mg to about 10,000 mg. In some embodiments, the first formulation comprises at least about 100, 150, 250, 500, 750, 1000, 1250, 2500, 3500, 4000, 5000, 6000 mg or 7000 mg of the flavonoid, or in a range of from one amount to another amount in the list of doses.

In some embodiments, the second formulation further comprises an NAD+ agonist, pharmaceutically acceptable salt, derivative or metabolite thereof. Suitable NAD+ agonists and concentrations thereof for use in the second formulation include those disclosed in Section II and IIIA herein.

### C. Additional Embodiments

In some embodiments, the formulations provided herein are essentially free of one or more preservatives, such as benzyl alcohol, phenol, m-cresol, chlorobutanol and benzethonium Cl. In other embodiments, a preservative may be included in the formulation, particularly where the formulation is a multidose formulation.

In some embodiment the formulations provided herein are pyrogen-free formulations which are substantially free of endotoxins and/or related pyrogenic substances. Endotoxins include toxins that are confined inside a microorganism and are released only when the microorganisms are broken down or die. Pyrogenic substances also include fever-inducing, thermostable substances (glycoproteins) from the outer membrane of bacteria and other microorganisms. Both of these substances can cause fever, hypotension and shock if administered to humans. In certain embodiments, the endotoxin and pyrogen levels in the composition are less than 10 EU/mg, or less than 5 EU/mg, or less than 1 EU/mg, or less than 0.1 EU/mg, or less than 0.01 EU/mg, or less than 0.001 EU/mg.

### V. Administration

The first and second formulations provided herein are administered to a mammal in need of treatment in accordance with known methods. Selection of a particular effective dose can be determined by a person skilled in the art based upon the consideration of several factors which will be known to the person skilled in the art, such as, the disorder to be treated, alleviated or improved; the nature and severity of the disorder being treated, the body mass of the host; and the like. The precise dose employed in the treatment; alleviation improvement of the disorder may also depend upon the route of administration. Effective doses can be extrapolated from dose-response curves derived from in vitro or animal model test systems. Dosage regimens comprising the formulations as described herein can be adjusted to provide the optimum desired response.

In some embodiments, the formulations can be prepared in unit dosage form for ease of administration and uniformity of dosage. “Unit dosage form,” as used herein refers to physically discrete units suited as unitary dosages for the subject to be treated; each unit dosage containing a predetermined quantity of the active ingredient is calculated to produce the desired effect in a formulation provided herein, e.g., a quantity calculated to provide an amount sufficient for a single cycle of administration.

The dosage regimen utilizing the formulations provided herein is selected in accordance with a variety of factors including type, species, age, weight, sex and medical condition of the patient; the severity of the condition to be treated; the route of administration; the renal or hepatic function of the patient; and the particular disclosed salt employed. A physician or veterinarian of ordinary skill in the art can readily determine and prescribe the effective amount of the drug required to prevent, counter or arrest the progress of the condition.

In some embodiments, a formulation comprising the NAD+ agonist is administered at a daily dose of between 1 mg/kg and about 500 mg/kg, between about 5 to about 500 mg/kg body weight; about 10 to about 200 mg/kg; about 10 to about 300 mg/kg; about 10 to about 200 mg/kg; or about 10 to about 100 mg/kg. In some embodiments, the NAD+ agonist is administered at a daily dose of about 20 mg/kg. In some embodiments, the NAD+ agonist is administered as a single dose. In other embodiments, the NAD+ agonist is administered in divided doses, e.g., two, three or four times a day, or in sustained release form. In certain embodiments, the NAD+ agonist is NaR or a pharmaceutically acceptable salt thereof. In certain embodiments, the NAD+ agonist is NaMN or a pharmaceutically acceptable salt thereof. In certain embodiments, the NAD+ agonist is NMN or a pharmaceutically acceptable salt thereof.

In some embodiments the flavonoid is administered at a dose of between about 5 mg/kg to about 100 mg/kg, about 10 mg/kg to about 100 mg/kg, about 20 mg/kg to about 100 mg/kg, about 20 mg/kg, about 25 mg/kg, about 30 mg/kg, about 40 mg/kg, about 50 mg/kg, about 60 mg/kg, about 70 mg/kg, about 75 mg/kg, about 80 mg/kg, about 90 mg/kg or about 100 mg/kg. In some embodiments, the flavonoid is administered at a dose of about 40 mg/kg. In certain embodiments, the flavonoid is selected from fisetin or geraldol. In some embodiments, the flavonoid is fisetin.

In some embodiments the flavonoid is administered for 2-3 consecutive days per week. In some embodiments the flavonoid is administered for 2-3 consecutive days every other week. In some embodiments, the flavonoid is administered for 2-3 consecutive days monthly. In some embodiments, the flavonoid is administered for 2 consecutive days monthly. In other embodiments the flavonoid is administered once per week, or twice per week. In some embodiments, the flavonoid is administered once every other week. In some embodiments, the flavonoid is administered once per month. In some embodiments, the flavonoid is administered twice per month.

In some embodiments, the administration of the first and second formulations comprises a monthly dosing regimen that is 28 days in length. In further embodiments, the administration of the second formulation occurs on the first and second days of the 28 days. The monthly dosing regimen may be repeated for as long as necessary to obtain the desired result. For example, the monthly dosing regimen may be repeated for at least 1 month, at least 2 months, at least 3 months, at least 4 months, at least 5 months, at least 6 months, at least 7 months, at least 8 months, at least 9 months, at least 10 months, at least 11 months, at least 12 months, at least 24 months, at least 36 months, at least 48 months, at least 60 months, at least 72 months, at least 84 months, at least 96 months, at least 108 months, at least 120 months, or at least 240 months.

In some embodiments, the second formulation comprises a flavonoid polyphenol and an NAD+ agonist. In some embodiments, the second formulation comprising the flavonoid and NAD+ agonist is administered for 2-3 consecutive days per week, followed by daily administration of the first formulation comprising the NAD+ agonist. In some embodiments the second formulation comprising the flavonoid polyphenol and the NAD+ agonist is administered for 2-3 consecutive days every other week, and the first formulation comprising the NAD+ agonist is administered daily for the remaining days each week. In some embodiments, the second formulation comprising the flavonoid is administered for 2-3 consecutive days monthly, and the first formulation comprising the NAD+ agonist is administered daily for the remaining days of the month. In other embodiments the second formulation comprising the flavonoid is administered once per week, or twice per week. In some embodiments, the flavonoid is administered once every other week. In some embodiments, the flavonoid is administered once per month. In some embodiments, the flavonoid is administered twice per month.

In certain embodiments, the first formulation comprises a salt of NaR and the second formulation comprises fisetin. In some embodiments, the first formulation comprises an inorganic salt of NaR and the second formulation comprises fisetin. In some embodiments, the first formulation comprises an amino acid salt of NaR and the second formulation comprises fisetin. In one embodiment, the first formulation comprises the tryptophan salt of NaR and the second formulation comprises fisetin.

In certain embodiments, the first formulation comprises a salt of NaMN and the second formulation comprises fisetin. In some embodiments, the first formulation comprises an inorganic salt of NaMN and the second formulation comprises fisetin. In some embodiments, the first formulation comprises an amino acid salt of NaMN and the second formulation comprises fisetin. In one embodiment, the first formulation comprises the tryptophan salt of NaMN and the second formulation comprises fisetin.

In certain embodiments, the first formulation comprises NaR or a pharmaceutically acceptable salt thereof and the second formulation comprises fisetin and NaR or a pharmaceutically acceptable salt thereof. In certain embodiments, the first formulation comprises an inorganic salt of NaR and the second formulation comprises fisetin and an inorganic salt of NaR. In certain embodiments, the first formulation comprises an amino acid salt of NaR and the second formulation comprises fisetin and an amino acid salt of NaR. In certain embodiments, the first formulation the tryptophan salt of NaR and the second formulation comprises fisetin and the tryptophan salt of NaR.

In certain embodiments, the first formulation comprises NaMN or pharmaceutically acceptable salt thereof and the second formulation comprises fisetin and NaMN or a pharmaceutically acceptable salt thereof. In certain embodiments, the first formulation comprises an inorganic salt of NaMN and the second formulation comprises fisetin and an inorganic salt of NaMN. In certain embodiments, the first formulation comprises an amino acid salt of NaMN and the second formulation comprises fisetin and an amino acid salt of NaMN. In certain embodiments, the first formulation comprises the tryptophan salt of NaMN and the second formulation comprises fisetin and the tryptophan salt of NaMN.

### VI. Kits and Articles of Manufacture

The formulations described herein also can be provided in a kit, a packaged combination of reagents in predetermined amounts with instructions for use in the methods described herein. For example, in some embodiments, the kits comprise a formulation as described herein include instructions, e.g., comprising administration parameters including dosages and dosage regimens.

Accordingly, provided herein is an article of manufacture with a container which holds the formulations disclosed herein. Suitable containers include, for example, bottles or blister packs. The container may be formed from a variety of materials such as glass or plastic. In some embodiments, the container excludes UV light.

In some embodiments, the container holds the formulation and the label on, or associated with, the container may indicate directions for use. The article of manufacture may further include other materials desirable from a commercial and user standpoint, including package inserts with instructions for use. Non-limiting examples of suitable instruction media include labels, pamphlets, inserts, and digital media.

### VII. Methods of Use

In some embodiments, provided herein is a method of treating or preventing a disease or disorder associated with aging, cellular degradation, and/or cellular restoration by administering to a subject in need thereof the first and second formulations provided herein.

Age-related disorders which can be treated according to the methods provided herein include loss of eye function, reduction in bone density, insulin insensitivity, frailty, osteoarthritis, cognitive dysfunction (e.g., dementia), metabolic disease (diabetes mellitus), obesity, inflammatory syndromes, cardiovascular disease, lipid disorders, age related muscle loss, osteoporosis, chronic kidney disease, vascular disease, or a combination thereof.

In some embodiments provided herein is a method of treating or preventing a neurogenerative disease or disorder by administering to a subject in need thereof first and second formulations provided herein.

Neurodegenerative diseases or disorders which can be treated according to the methods provided herein include disorders which impair cognitive performance, e.g., a neurodegenerative disease or a central nervous system condition, such as a disease selected from the group consisting of Lewy body diseases, Alzheimer's disease, amnio tropic lateral sclerosis (ALS), Parkinson's Disease, Huntington's Chorea, senile dementia, Pick's disease, parkinsonism dementia syndrome, progressive subcortical gliosis, progressive supranuclear palsy, thalamic degeneration syndrome, hereditary aphasia, and myo-clonus epilepsy. Other diseases that may be treated include a condition or disease selected from the group consisting of frontal-temporal dementia, mood and anxiety disorders, depression, Schizophrenia, autism, anxiety, panic attacks, binge eating, social phobia, an affective disorder, a psychiatric disorder, mild cognitive impairment, seizures, neurodegenerative illnesses, dementia, head trauma or injury, hysteria accompanied by confusion, cognitive disorders; age-related dementia; age-induced memory impairment; ion deficit disorder; psychosis; cognitive deficits associated with psychosis; and drug.

The efficacy of the formulations and methods provide herein can be determined by improvements in one or more of a variety of behaviors, for example, walking endurance, gait speed, increased strength, improved cardiovascular activity (e.g., CV stress test) and heart rate. In some embodiments, the efficacy of the formulations and methods provided herein also can be determined by assessment of the Frailty Index (FI) of the subject before treatment, during the course of treatment at different time points (e.g., after one week, one month, six months, etc.) and after treatment (e.g., Kane et al., J. Gerontol. A. Biol. Sci. Med. Sci. 71(3):333-339, 2016; Liu et al., J. Gerontol. A. Biol. Sci. Med. Sci. 69(12):1485-1491).

In addition, a variety of markers can be used as surrogates for monitoring the efficacy of the methods provided herein, using methods known in the art such as bioassays, immunoassays, flow cytometry and assays utilizing nanoparticle-modified aptamers. Suitable markers include but are not limited to, for example, the measurement inflammatory cytokines (TNF, IL-1, IL-6) (e.g., reviewed in Shokrani, 2011, from https://www.mlo-online.com/home/article/13004250/cytokines-utility-and-laboratory-measurement), DNA methylation levels, improvements in lipid metabolism, and insulin sensitivity (e.g., HbA1C levels) and lactate metabolism as a measure of cellular metabolism and exercise tolerance (Gladden, J. Physiol. 558.1:5-30, 2004).

The following examples are merely illustrative and should not be construed as limiting the scope of this disclosure in any way as many variations and equivalents will become apparent to those skilled in the art upon reading the present disclosure.

### EXAMPLES

### Example 1—Seven Day Repeat Dose Tolerability Study of NaR

The objective of this study was to evaluate the tolerability and pharmacokinetic profile following 7-days of repeat dose administration of NaR.

### Materials and Methods

NaR is provided as 100 mg capsule, size 4, stored at 18-26° C., and fisetin is provided as 100 mg capsule, size 0, stored at 18-26° C. Canis familaris (dog)/Beagle—nine non-naïve males, age 1-3 years, weight 7-11 kg.

The following table presents the study group arrangement:

| | | | Dosage | Capsules Dosed × |
|---|---|---|---|---|
| Group | No. of | Test | Level | Concentration |
| No. | Males | Article | (mg/kg/day) | (number × mg) |
| 1 | 3 | NaR | 10 | 1 × 100 |
| 2 | 3 | NaR | 20 | 2 × 100 |
| 3 | 3 | NaR | 30 | 3 × 100 |

The appropriate number of NaR capsules were administered once daily to non-naïve male beagle dogs. Capsules were followed by at least 5 mL of tap water to ensure complete delivery. Animals were fed approximately 2 hours prior to dosing, with food removed approximately 2 hours post-dose. Samples for bioanalysis were collected during the pre-dose period (Day −7; based on Time Zero of 9 am) at 0, 0.25, 0.5, 1, 2, 4, 6, 8, 12, and 24 hours post-Time Zero. Samples were collected on Day 1 and 7 at 0, 0.25, 0.5, 1, 2, 4, 6, 8, 12, and 24 hours post dose, and on Days 3-5 at 2 hours post dose. Samples were analyzed for NAD+ using a bioassay.

### Results

There were no test article-related effects on mortality or moribundity, clinical observations, body weight, or food consumption. All animals had measurable endogenous levels of NAD+. Median Tₘₐₓ were reached in less than 2 hours post dose in all animals, with values ranging between 0 and 2 hours. Mean Cₘₐₓ for all dose groups showed accumulation between Day 1 and 7. This difference was notably higher for the highest dose group (30 mg/kg/day). Mean AUC₀₋₂₄ and partial AUCs (0-8 and 8-24) showed approximately the same trend as Cₘₐₓ.

Due to wide range in endogenous levels, Cₘₐₓ and AUC₀₋₂₄ ratios between Day 7 and Day 1 for each individual animal was compared. Cₘₐₓ shows a clear increase in the highest dose group as compared to the others. The AUC₀₋₂₄, however, reached a higher value in the mid dose group (20 mg/kg/day) than the other two groups.

Based on the results of this study, capsule dose administration of NaR to male Beagle dogs for seven days at 10, 20, and 30 mg/kg/day was well tolerated. No test article-related effects were observed on mortality or moribundity, clinical observations, body weights, or food consumption. Based on the results of bioanalysis, oral capsule administration of NaR resulted in measurable increases in NAD+ levels and therefore is considered bioavailable.

### Example 2—Tolerability of Orally Administered NaR

The objective of the study described in this example is to evaluate the tolerability of NaR administered orally.

### Materials and Methods

The tolerability of NaR administered orally was determined in a two-phase laboratory study in purpose-bred beagles (male and female, 6.3-9.2 kg, 6 months old at the start of the study). The first phase of the study included single dose escalations of 100, 300, 1000 and 2000 mg/kg (n=4 per group). The second phase tested repeat dosing consisting of daily dosing over seven days of 100, 300 and 1000 mg/kg (n=6 per group). Administration occurred via oral gavage with an 18 French catheter into the stomach. The animals were assigned to groups by a stratified randomization scheme designed to achieve similar group mean body weights. Males and females were randomized separately. All animals survived the study.

### Results

There were no treatment related clinical observations. Body weights and food consumption were unaffected by treatment. A single clinicopathologic finding was noted. Potential test article-related increased mean sorbitol dehydrogenase (SDH) concentrations were noted in the 300 and 1000 mg/kg group males and 300 mg/kg group females on Day 2 compared to pretreatment period values. The increase in SDH activity was considered non-adverse because of the low magnitude in effect (e.g., 0 U/L to 2 U/L), and because it had no effect on other liver enzymes. Even though the changes were minimal and were within the laboratory's historical background range, the trends pointed to a potential test article-related effect. There were no other test article-related effects on serum chemistry. The maximum tolerated dosages were the highest dosages tested, 2000 mg/kg for the single dosing phase and 1000 mg/kg/day for the 7-day repeat dosing phase.

### Example 3—Activity and Safety of Orally Administered NaR

The objectives of the study described in this example is to evaluate the activity and safety of NaR administered orally.

### Materials and Methods

A double blinded, placebo-controlled, non-randomized laboratory study to assess activity and safety was completed in 15 mature purpose-bred beagles and mongrels over a period of 28 days. The four males and 11 females ranged in age from 4-11 years, with a mean of 7.75 years. Body weights for beagles (n=12) were 9.5-15 kg and for mongrels (n=3) 24-40 kg. The animals were fitted with activity collars and housed in a room that allowed free movement for ˜ nine hours per day. They received three enrichment periods of 15 minutes per day consisting of interaction with caretakers. The activity of the animals was tracked 24 hours per day using actigraphy (accelerometers attached to the animal's collar) and force plate analysis for assessing changes to gait. A modified canine cognitive dysfunction rating (CCDR) (Madari et al., “Assessment of Severity and Progression of Canine Cognitive Dysfunction Syndrome Using the Canine Dementia Scale (CADES),” Applied Animal Behaviour Science 171: 138-145 (2015), which is hereby incorporated by reference in its entirety) and canine orthopedic index (COI) (Brown, D., “The Canine Orthopedic Index. Step 1: Devising the Items,” Veterinary Surgery 43: 232-240 (2014); Brown, D., “The Canine Orthopedic Index. Step 2: Psychometric Testing,” Veterinary Surgery 43: 241-246 (2014); and Brown, D., “The Canine Orthopedic Index. Step 3: Responsiveness Testing,” Veterinary Surgery 43: 247-254 (2014), which are hereby incorporated by reference in their entirety) were completed pretreatment, and on days 14 and 28. Physical examination, body weight, body condition score (BCS) and complete blood count and blood chemistry were evaluated pretreatment and weekly. The animals were dosed orally with capsules daily after an overnight fast.

### Results

Compared with controls, a positive effect was seen in the diurnal rhythm of treated dogs, with an increase in overall activity (frequency and intensity) and less movement when resting (deeper sleep). No adverse clinical observations were noted and blood chemistry and hematology results remained within the reference ranges. Minor changes in body weight and BCS occurred in both treated and control groups, without trends. No changes were noted in CCDR and COI over the course of the study.

### Example 4—NAD+ Concentrations and Stability in in Whole Blood Samples

Concentrations for NAD+ were analyzed and concentrations of NAD+ were analyzed and long-term stability assays of NAD+ were performed over a 1 week and 1 month long period. Samples were stored at −70° C. for the duration of this stability study. A simple test against control experiment was performed at both 1 week and 1-month time points.

Freshly treated dog whole blood samples were aliquoted and placed in the −70° C. freezer for all time points and compared against a freshly prepared curve of treated DiH20 containing fresh true QCs for consistency. A treated sample was also aliquoted and placed in the −20° C. freezer for 1 month and compared against a control. For the results to be accepted the percent difference of test vs. control was required to be ±20.0%.

### Non-Compartmental Pharmacokinetics

A non-compartmental Pharmacokinetic (PK) approach consistent with oral route of administration was used to estimate PK parameters in Watson LIMS (version 7.5). All parameters were generated from individual concentrations in treated whole blood whenever practical. Mean values for each dose group were also reported. All concentration values were in ng/mL, and all time points were in hours. Nominal dose concentrations and sampling times were used. The observed maximum treated whole blood concentration (Cmax), time of Cmax (Tmax), and AUC (area under the concentration vs. time curve) were determined directly from the data during the entire 24 hour time interval post dosing. The dose levels were either 10, 20, or 30 mg/kg/day (Groups 1, 2, and 3, respectively). PK samples were collected on days −7 predose, 1 and 7 post dose, of a seven day once daily study. Samples on days 3, 4, and 5 postdose, at only one timepoint were collected but not included in PK parameter estimation.

Whenever possible, standard deviation and coefficient of variation determined by Watson LIMS were used. For analyses that were not supported by Watson, i.e. custom comparisons, Excel was used, and the statistics were reported as determined by Excel algorithms.

### Results

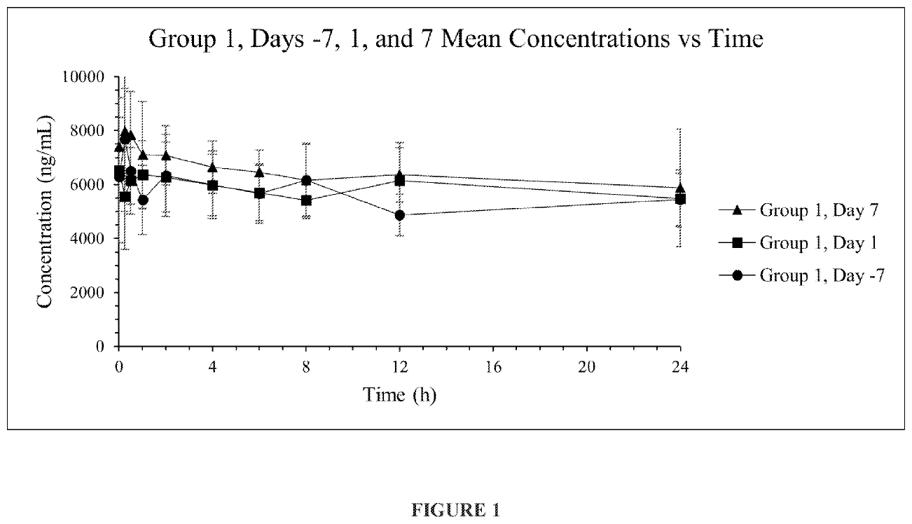

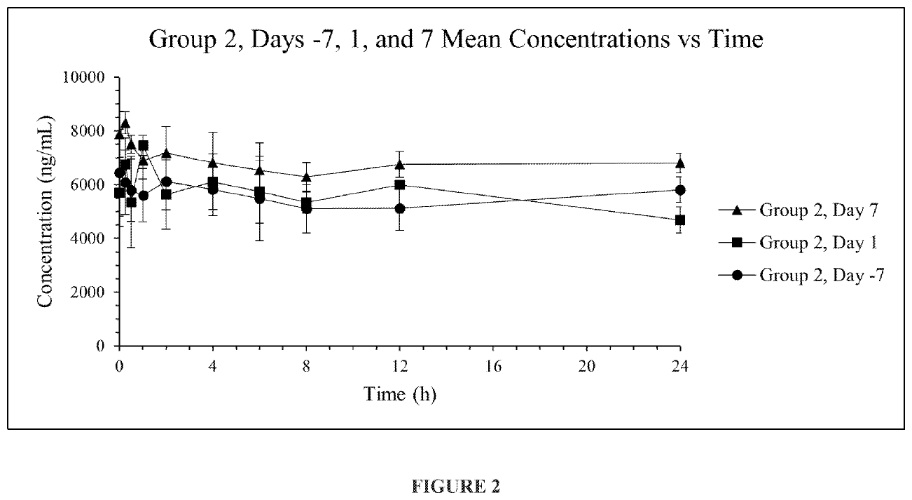

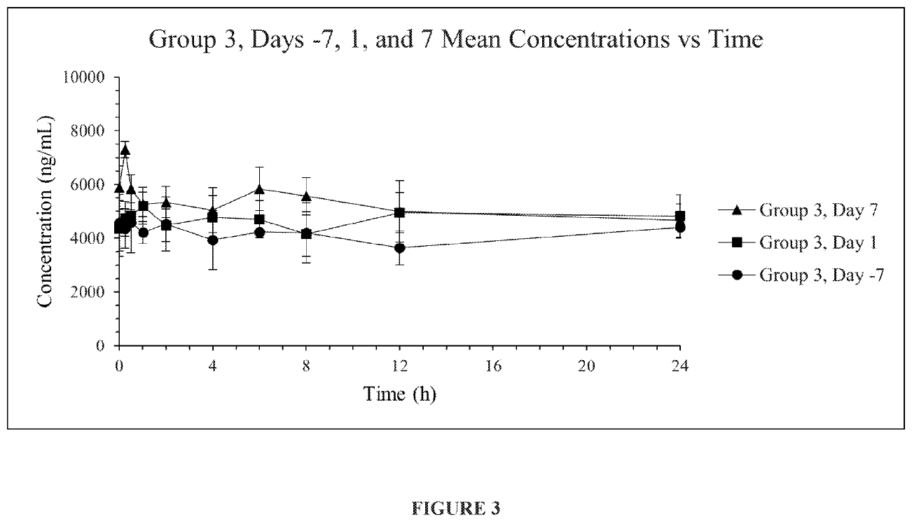

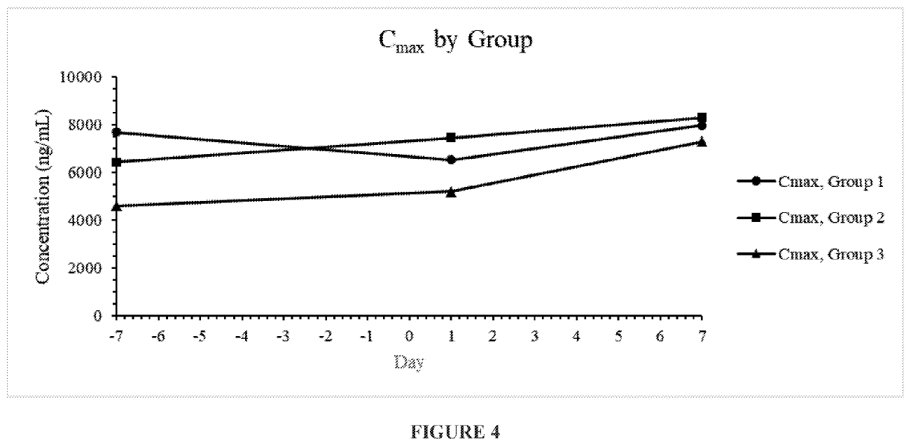

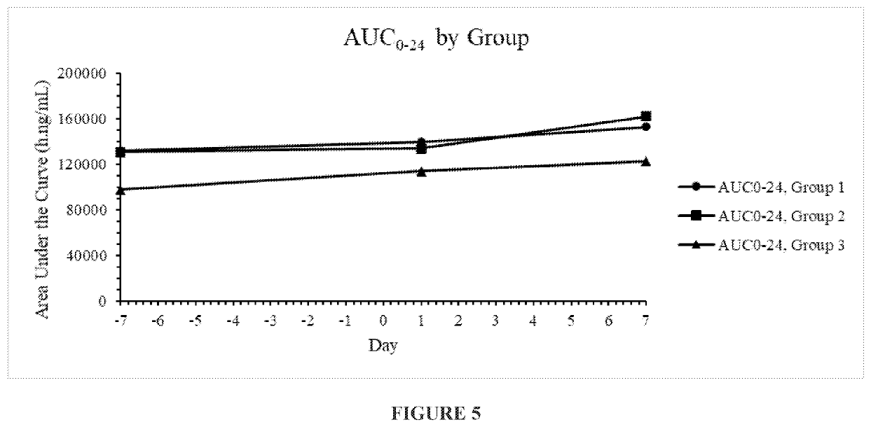

Treated whole blood concentration versus time course graphs are in FIG. 1 through FIG. 3. Median Tₘₐₓ were reached in less than 2 hours post dose. The values ranged between 0 and 2 hours. Mean Cₘₐₓ for all dose groups showed an increase between day 1 and 7. This difference was significantly higher for the highest dose groups (30 mg/kg/day). See FIG. 4.

Mean AUC₀₋₂₄ follows the same trend as mean Cₘₐₓ. Partial AUCs (0-8 and 8-24) were also measured and showed approximately the same trend as AUC₀₋₂₄. See FIG. 5 for data.

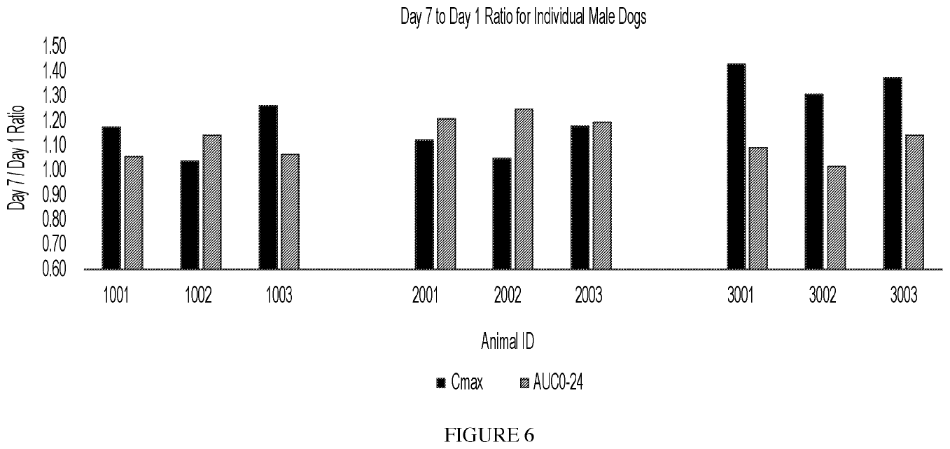

Due to wide range in endogenous levels, Cmax and AUC₀₋₂₄ ratios between Day 7 and Day 1 for each individual animals were compared. Cmax shows a clear increase in the highest dose group as compared to the others. The AUC₀₋₂₄, however, reaches a higher value in the mid dose group (20 mg/kg/day) than the other two groups. See FIG. 6.

### Example 5—Frailty Measurements in Aged C56BL/6 Male Mice

Frailty was measured in aged C56BL/6 male mice, starting at 18 months old (n=50 per group) monthly, over time compared with placebo-treated controls. NaR was administered in the water in low and high dosages of 60 and 400 mg/kg. The animals were group housed 4 per cage and the water was changed weekly. The animals were fed ad libitum and received daily photoperiods of approximately 12 hours of light alternating with 12 hours of darkness.

Three validated scoring methods, Frailty Index (Whitehead et al., “A clinical frailty index in aging mice: Comparisons with frailty index data in humans” J Gerontol A Biol Sci Med Sci. 69:621-632(2014)), FRIGHT Age (Schultz et al., “Age and life expectancy clocks based on machine learning analysis of mouse frailty” Nature Comm. 11(1):4618 (2020)) and AFRAID score (Schultz et al., “Age and life expectancy clocks based on machine learning analysis of mouse frailty” Nature Comm. 11(1):4618 (2020)) were conducted at 25 months of age. The clinical Frailty Score includes non-invasive measures of potential health deficits: general discomfort, piloerection (bristling of hair), grimace (facial expression), breathing rate, eyes (cataracts, corneal opacity, discharge, size), whiskers (color and number), reflex blinking of eyes, gait, muscle tremor, hair loss, fur color loss, growths, rectal prolapse, evidence of diarrhea, distention of abdomen, body condition, spinal curvature, occlusion of teeth, nasal discharge, body weight. Each measure is assigned 0, 0.5 or 1 for not present, mildly frail and most frail, respectively. FRIGHT age (Frailty Inferred Geriatric Health Timeline) is a measure of a mouse's apparent chronological age and the AFRAID clock (Analysis of Frailty and Death) is a prediction of a mouse's life expectancy

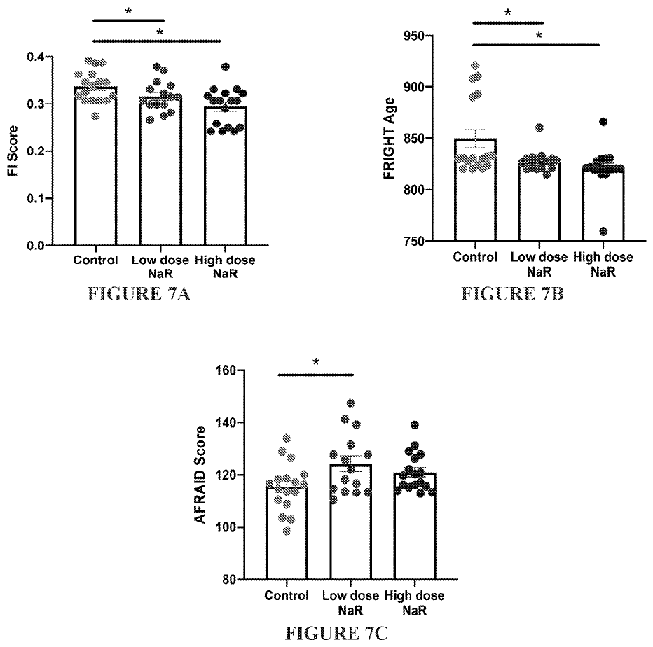

As measured by all three methods, the low dose NaR-treated mice were significantly less frail than controls at the end of the study (FIGS. 7A-7C) (a statistical significance of p<0.05 calculated using a one-way ANOVA then a Tukey's multiple comparisons test). In two of the three methods, both treatment groups were significantly different compared with controls.

### Example 6—Bioavailability and Pharmacokinetics of NaR and Fisetin

The objectives of the studies described in this example is to evaluate the bioavailability of a series of dosages and dosing regimens of NaR and Fisetin.

### Materials and Methods

A series of pharmacokinetic studies were conducted to evaluate the bioavailability of a series of dosages and dosing regimens as measured in whole blood in non-fasted, adult Beagle dogs. NaR, NMN, Fisetin, individually and combination dosing (NaR-Fisetin and NMN-Fisetin) were tested.

Tables 1 and 2 show the dosing regimens of Study #1 conducted over 7 days and Study #2 conducted over 28 days, respectively. In Study #1, three dosage combinations were tested (Groups 1-3). An additional group (Group 4) was included to test the effects of fasting on the whole blood concentration of NAD. In Study #2, the effects of each compound individually and in combination were evaluated (Groups 1-3). Study #2 contained another test group (Group 4), which was included to compare the effects of NMN vs NaR in combination with Fisetin by Day 7 to determine if the effects seen on NAD concentration were unique to NaR vs. NAD booster, in general. Baseline NAD concentrations sampled over 24 hours on day −7 served as controls for endogenous NAD concentrations.

TABLE 1
Dosing Regimen - Study #1

| | | | | | Fisetin | | |
|---|---|---|---|---|---|---|---|
| | | | | NaR Capsules | Capsules | | |
| Group | No. of | Test | Dosage Level | to Be Dosed | to Be Dosed | | |
| No. | Males | Articles | (mg/kg/day) | (mg) | (mg) | Feeding Schedule | Dosing Schedule |
| 1 | 3 | NaR + | 10 mg/kg NaR + | 1 × 100 | 2 × 100 | ~2 h predose to | NaR: Days 1-7 |
| | | Fisetin | 20 mg/kg Fisetin | | | ~2 h postdose | Fisetin: Days 1-2 |
| 2 | 3 | NaR + | 20 mg/kg NaR + | 2 × 100 | 4 × 100 | ~2 h predose to | NaR: Days 1-7 |
| | | Fisetin | 40 mg/kg Fisetin | | | ~2 h postdose | Fisetin: Days 1-2 |
| 3 | 3 | NaR + | 30 mg/kg NaR + | 3 × 100 | 6 × 100 | ~2 h predose to | NaR: Days 1-7 |
| | | Fisetin | 60 mg/kg Fisetin | | | ~2 h postdose | Fisetin: Days 1-2 |
| 4 | 3 | NaR + | 30 mg/kg NaR + | 3 × 100 | 6 × 100 | ~4 h postdose | NaR: Days 1-7 |
| | | Fisetin | 60 mg/kg Fisetin | | | (fasted pre-dosing) | Fisetin: Days 1-2 |

TABLE 2
Dosing Regimen - Study #2

| | | | | No. Capsules | | |
|---|---|---|---|---|---|---|
| Group | No. of | Test | Dosage Level | to Be Dosed | | |
| No. | Males | Article(s) | (mg/kg/day) | (mg) | Feeding Schedule | Dosing Schedule |
| 1 | 3 | NaR | 20 mg/kg NaR | NaR: 2 × 100 | ~2 h predose to | Days 1-28 |
| | | | | | ~2 h postdose | |
| 2 | 3 | Fisetin | 20 mg/kg Fisetin | Fisetin: 2 × 100 | ~2 h predose to | Days 1-2 |
| | | | | | ~2 h postdose | |
| 3 | 3 | NaR + | 20 mg/kg NaR + | NaR: 2 × 100 | ~2 h predose to | NaR: Days 1-28 |
| | | Fisetin | 20 mg/kg Fisetin | Fisetin: 2 × 100 | ~2 h postdose | Fisetin: Days 1-2 |
| 4 | 3 | NNM + | 20 mg/kg NMN + | NNM: 2 × 100 | ~2 h predose to | NMN: Days 1-7 |
| | | Fisetin | 20 mg/kg Fisetin | Fisetin: 2 × 100 | ~2 h postdose | Fisetin: Days 1-2 |

### Test Articles for Study #1 and Study #2:

NaR was provided as 100 mg capsule, fisetin was provided as 100 mg capsule, and NMN was provided as 100 mg capsule. All capsules were stored at ambient temperature (i.e., 18-26° C.).

### Animals for Study #1 and Study #2:

Canis familaris (dog)/Beagle—twelve non-naïve males, age 1-3 years, weight 7-11 kg. This species and strain of animal is recognized to be appropriate for tolerability studies. The dogs were individually housed in stainless-steel cages during periods of food consumption measurement and as needed during the PK collection periods. All animals were given regular opportunity for exercise and environmental enrichment was provided.

The temperature and humidity in the room was continuously monitored and regulated, and the animals received daily photoperiods of approximately 12 hours of light alternating with 12 hours of darkness.

A canine diet was offered once daily (approximately 250 grams/animal/day). Water was available ad libitum.

### Dosing Schedule for Study #1:

Days 1-2: The appropriate number of NaR capsules and Fisetin capsules were administered once daily (See Table 1), followed by at least 5 mL of tap water to ensure complete delivery.

Days 3-7: The appropriate number of NaR capsules were administered once daily (See Table 1), followed by at least 5 mL of tap water to ensure complete delivery.

In instances where a whole capsule was regurgitated, the animal was re-dosed with a new capsule and the time recorded.

### Dosing Schedule for Study #2:

The appropriate number of NaR capsules Fisetin capsules and/or NMN capsules were administered once daily (See Table 2), followed by at least 5 mL of water to ensure complete delivery.

In instances where a whole capsule was regurgitated, the animal was re-dosed with a new capsule and the time recorded.

### Feeding Schedule for Study #1:

The feeding schedules were followed for the pre-dose blood collections beginning on Day −2 and continued through Day 8.

Groups 1-3: Food was offered once daily, approximately 2 hours prior to dose administration. Food was removed approximately 2 hours post-dose.

Group 4: Food was offered once daily, approximately 4 hours post-dose, i.e., the dogs were administered the test articles in a fasted state.

### Feeding Schedule for Study #2:

Food was offered once daily, approximately 2 hours prior to dose administration. Food was removed least 2 hours post-dose.

### Clinical Observations for Study #1:

Daily observations occurred on the days of the pre-dose blood collection.

Days 1-7: The animals were observed daily, prior to dose and 1-2 hours post-dose. Their food intake was measured beginning on Day −2 and continued through Day 8.

### Clinical Observations for Study #2:

Days 1-28: The animals were observed daily, prior to dose if applicable. Their food intake was measured beginning on Day 0 and continued through Day 28. The animal's body weight was measured on Day 1 and at least twice weekly thereafter, and on Day 28.

### Pharmacokinetic Evaluation Study #1:

Two mL blood samples were collected at each time point using K₂EDTA as an anticoagulant from all animals in each dose group. A sample was obtained prior to dosing (Day −7 pre-dose). On Day −7, groups 1-3 had 1 sample collected prior to feeding, and at times 0, 0.25, 0.5, 1, 2, 4, 6, 8, 12, and 24 hours post-Time Zero. On Day 2, groups 1-3 had 1 sample collected prior to feeding (˜2 hr prior to Time Zero) and at times 0, 0.25, 0.5, 1, 2, 4, 6, 8, 12, and 24 hours post-dose. On Days 3-5, samples were collected 2 hours post-dose, and on Day 7 samples were collected prior to feeding Groups 1-3 (˜2 hours prior to Time Zero) from all animals, and at time points 0.25, 0.5, 1, 2, 4, 6, 8, 12, and 24 hours post-dose.

For Group 2, on Day 21, post-dosing, and on Day 35, one sample was collected prior to feeding, and at times 0, 0.25, 0.5, 1, 2, 4, 6, 8, 12, and 24 hours post-Time Zero. The samples were processed for NAD+.

### Pharmacokinetic Evaluation Study #2:

Two mL blood samples were collected at each time point using K₂EDTA as an anticoagulant from all animals in each dose group. On Day 0 each group had 1 sample collected at times 0, 0.5, 1, 2, 4, 8, and 12 hours post-Time Zero. On Days 1, 2, 3, and 7, each group had 1 sample collected at times 0, 0.5, 1, 2, 4, 8, and 12 hours post-dose. On Days 14, 21, and 28 Groups 1-3 had samples taken at 0, 0.5, 1, 2, and 12 hours post-dose.

### Sample Handling for Study #1 and Study #2

Immediately after collection, the sample tubes were inverted multiple times to ensure homogenization, then placed in an ice water bath until analysis. All samples were processed for NAD+ analysis and Fisetin analysis.

### Pharmacokinetic Evaluation for Study #1 and Study #2

The bioanalytical data was used in the generation of a pharmacokinetic data using parameters calculated for each individual animal for Days 2 and 7, including: Tmax, Cmax, AUC₀₋₂₄ and T½, as described in the previous examples, and the effect of repeat dosing on exposure was assessed by statistical analyses, e.g., mean and standard deviation for numerical parameters.

### Results

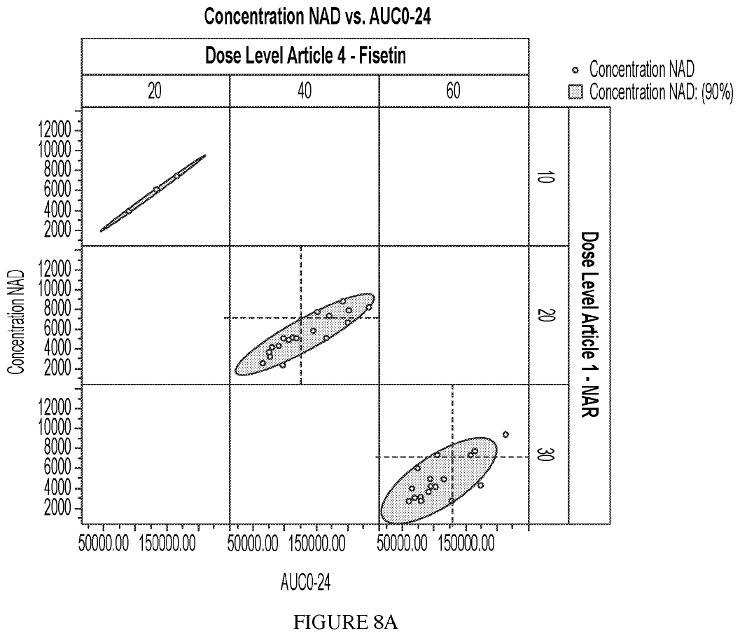

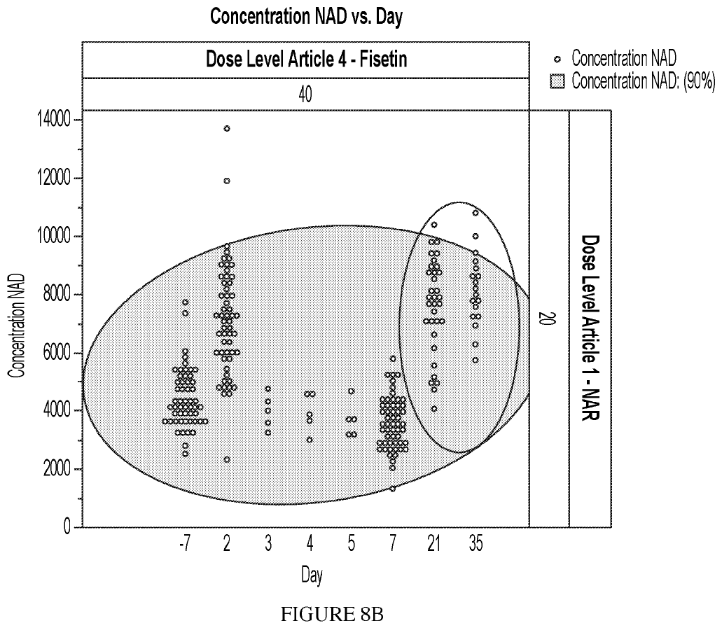

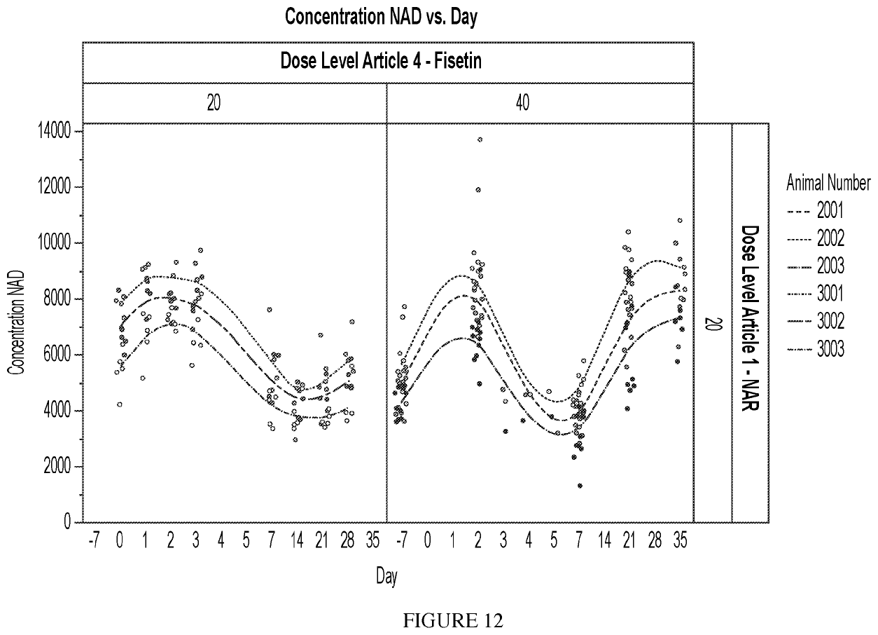

Overall, a dose effect was observed with NaR and fisetin; fasted dogs had lower concentrations of NAD pre and post treatment. No significant differences in NAD concentrations were found between combinations of NMN and Fisetin, and NaR and Fisetin, after 7 days of dosing. A monthly dosing schedule of 20 mg/kg NaR (daily) and 40 mg/kg Fisetin (Days 1 and 2 only) showed the optimum results based on data presented in FIGS. 8-12. Surprisingly, the administration of 20 mg/kg NaR (daily) and 40 mg/kg Fisetin (Days 1 and 2 only) resulted in a decrease in NAD concentration on days 7 and 14, after the expected increase which occurred on day 2. However, after this transient decrease, the NAD concentration subsequently increased above baseline values on days 28 and 35 (FIG. 12).

In Study #1, the dosing of NaR 20 mg/kg with fisetin 40 mg/kg produced the largest subject population with the highest concentrations of NAD over the course of 24 hours (FIG. 8A). FIG. 8A is a correlation plot of whole blood NAD concentrations and AUC₀₋₂₄ for the differing NaR and Fisetin dose levels tested. A comparison of the overall exposure (AUC₀₋₂₄) with the NAD concentration confirmed that an increased NAD concentration resulted a higher overall exposure. Since the same scale is used in each box in FIG. 8A, data points left to right show greater exposure and data points bottom to top show increased concentration. In other words, the desired outcome is to observe a higher number of data points in the upper right quadrant of each box. On this basis, the combination dosage of 20 mg/kg NaR (daily) and 40 mg/kg fisetin (on days 1, and 2 only) is shown to be the optimal dosage. Note that the complete data set is represented and the data points in the lower left quadrant include any condition that resulted in lower NAD concentrations (such as pre-dose, and non-peak days).

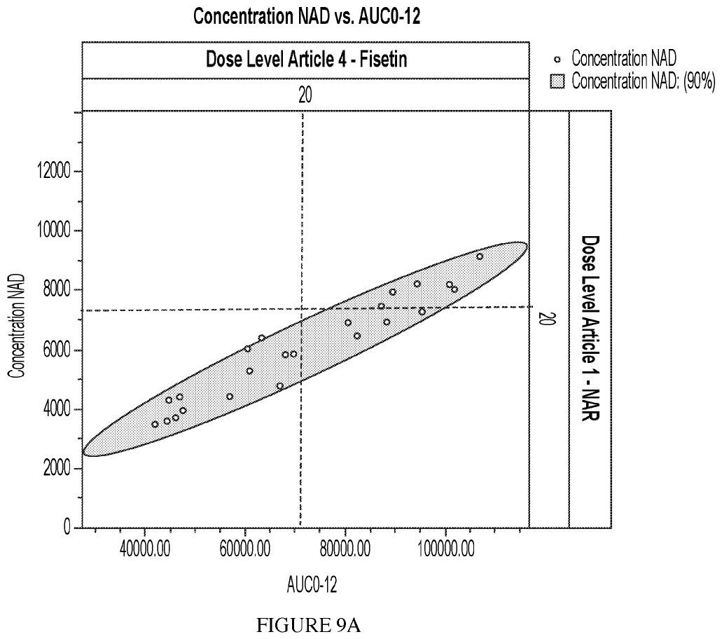

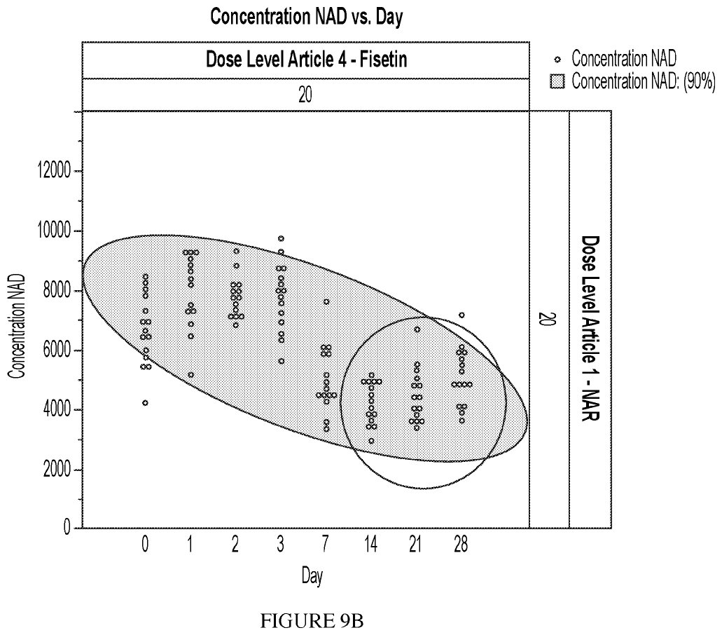

Furthermore, peak concentrations of NAD were observed on day 2 in all dosing groups despite daily dosing of NaR through day 7. Based on this finding, NAD concentrations were measured on days 21 and 35 in dogs which displayed the greatest increase in NAD concentrations, (Group 2-20 mg/kg NaR+40 mg/kg fisetin; FIG. 8B). Following a peak concentration on Day 2, NAD levels decreased to baseline values on days 3, 4, 5 and 7 despite daily dosing with NaR. Without further administration of NaR or fisetin, NAD levels then increased on days 21 and 35 to the peak levels previously seen on Day 2. This increase in NAD concentration on days 21 and 35 is not observed with other dosing regimens. For example, in Study #2, the dosing of NaR 20 mg/kg with fisetin 20 mg/kg produced NAD concentrations over the course of 12 hours (AUC₀₋₁₂) on days 14, 21 and 28 that do not recover to the higher concentrations obtained on previous days (FIGS. 9A-9B).

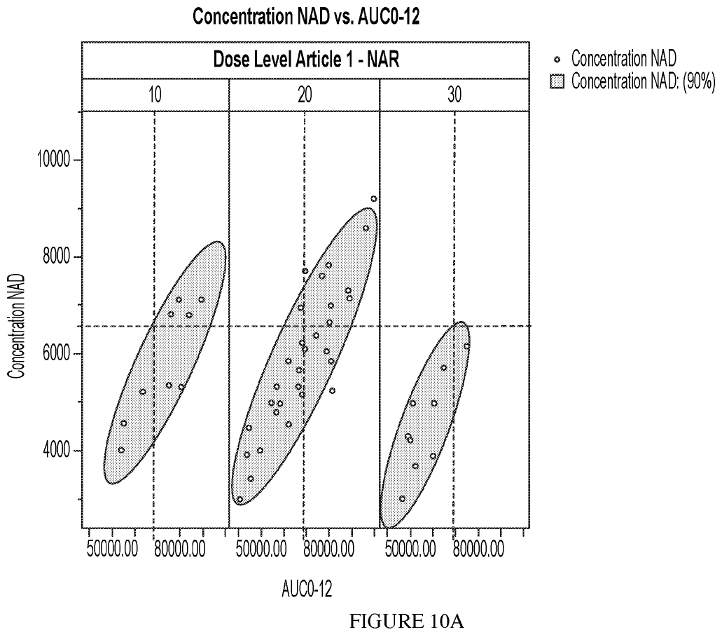

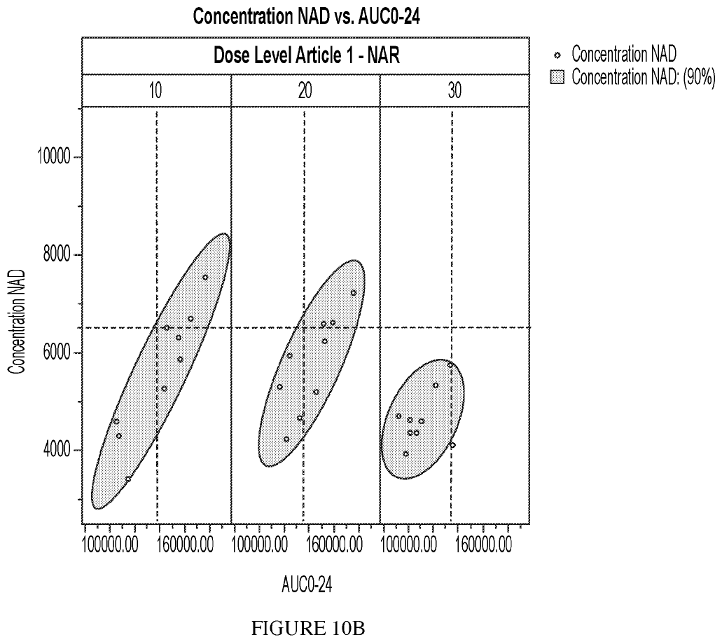

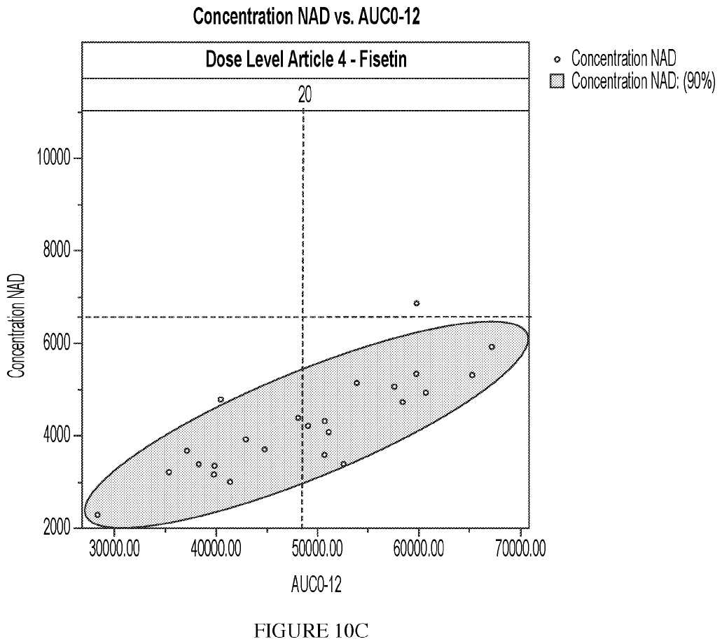

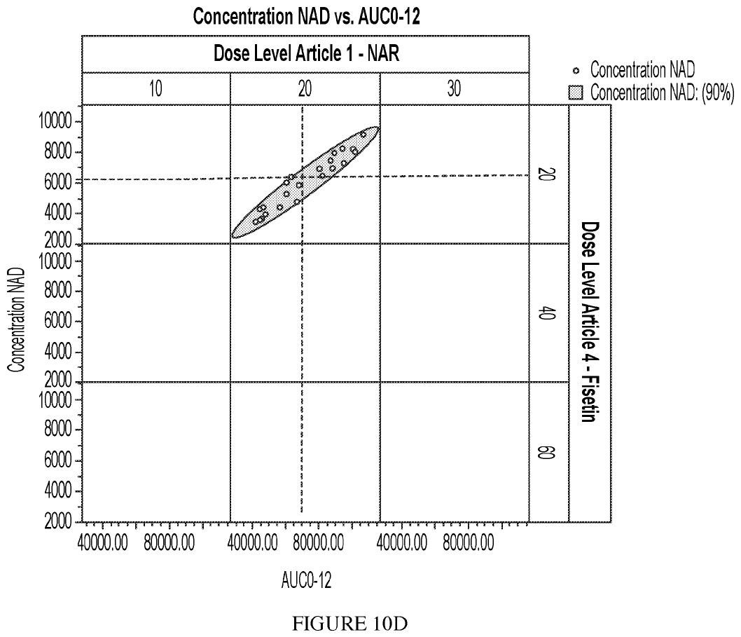

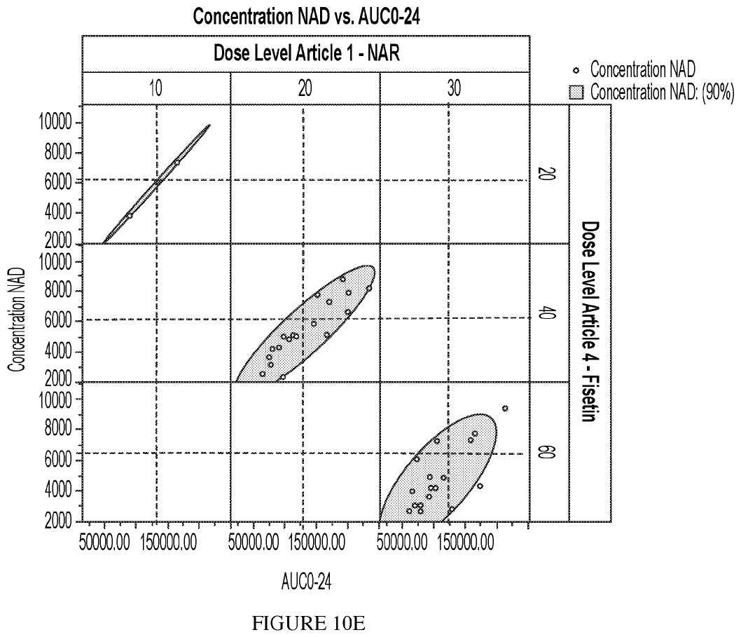

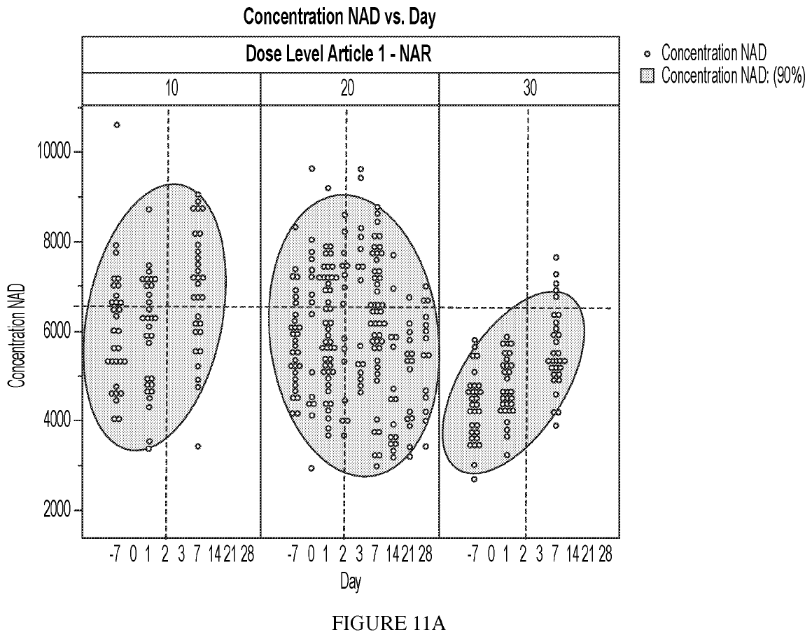

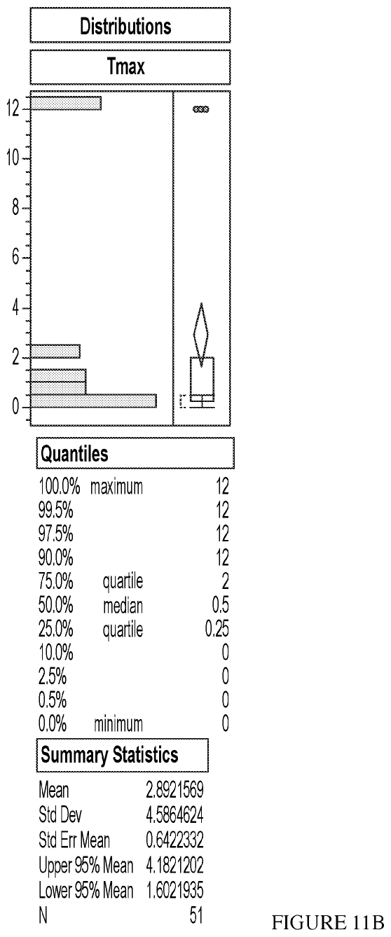

FIGS. 10A-10E show the comparison of NAD concentrations (ng/mL) and total exposure (AUC₀₋₁₂ and AUC₀₋₂₄) for all of the dosings in both Study #1 and Study #2. FIGS. 10A and 10B display the NAD concentration obtained from administration of only NaR, with a dose of 20 mg/kg producing the highest AUC and NAD concentrations for this subset of subjects. FIG. 10C displays the NAD concentration obtained from administration of only fisetin. The concentration of NAD remained low with fisetin only dosing (below 7000 ng/mL). FIGS. 10D and 10E display the NAD concentration obtained from the combined dosing of NAR and fisetin. The dosing of 20 mg/kg NAR with 20 mg/kg fisetin produced majority of days in the upper right-hand quadrant; however, the AUC is lower than that of the 20 mg/kg NAR and 40 mg/kg fisetin dose level (AUC₀₋₁₂: 100,000 vs AUC₀₋₂₄: 160,000). These data indicate the optimal dose is 20 mg/kg NAR and 40 mg/kg fisetin.

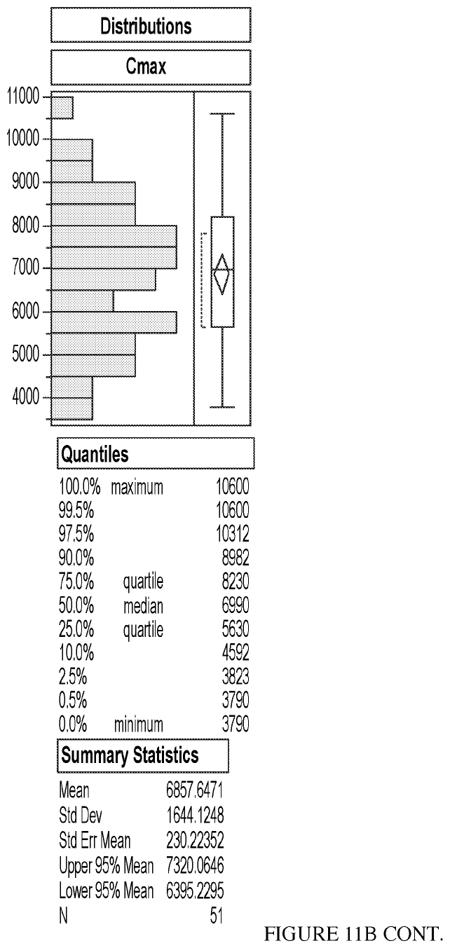

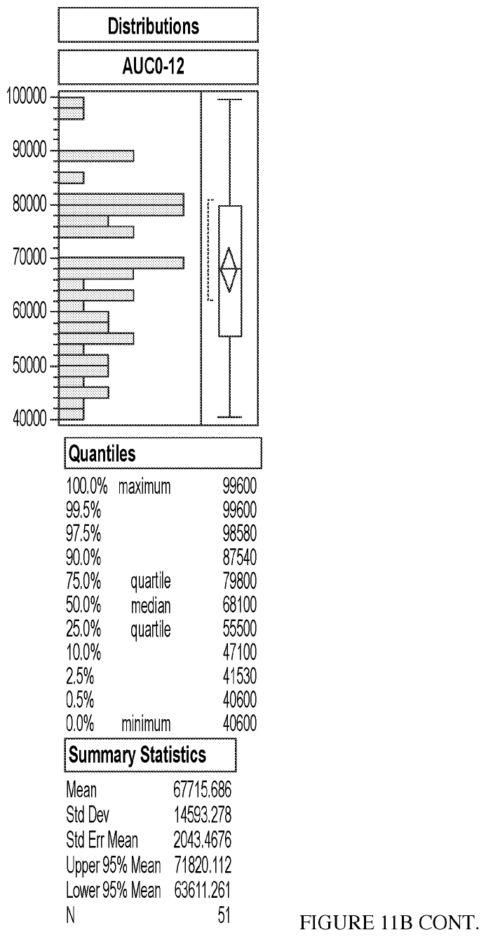

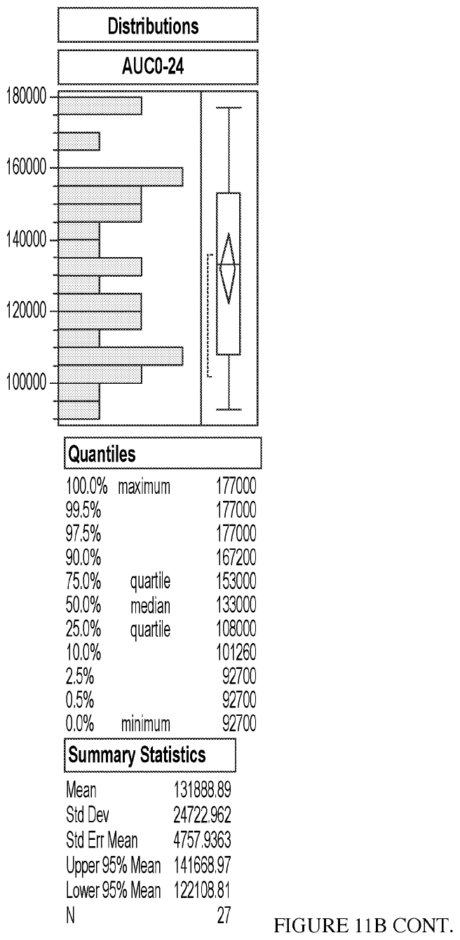

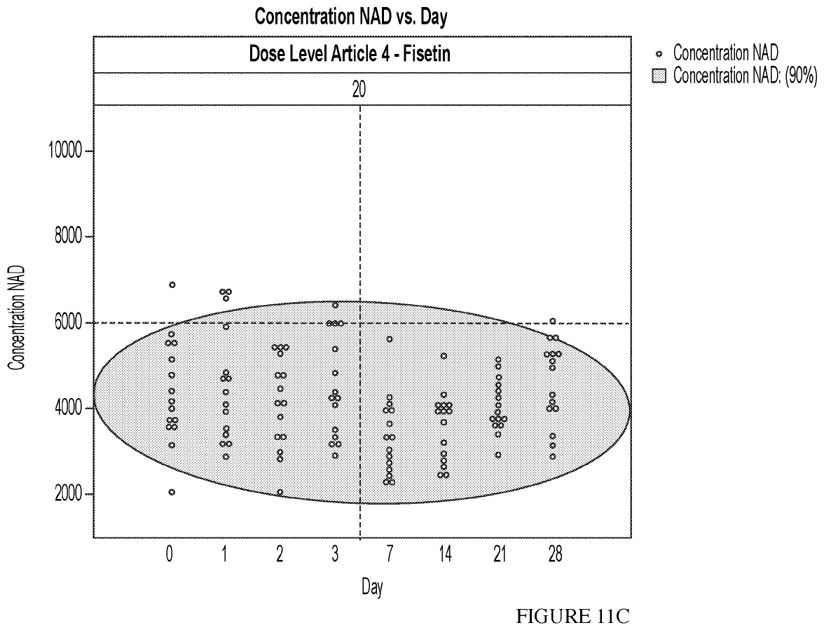

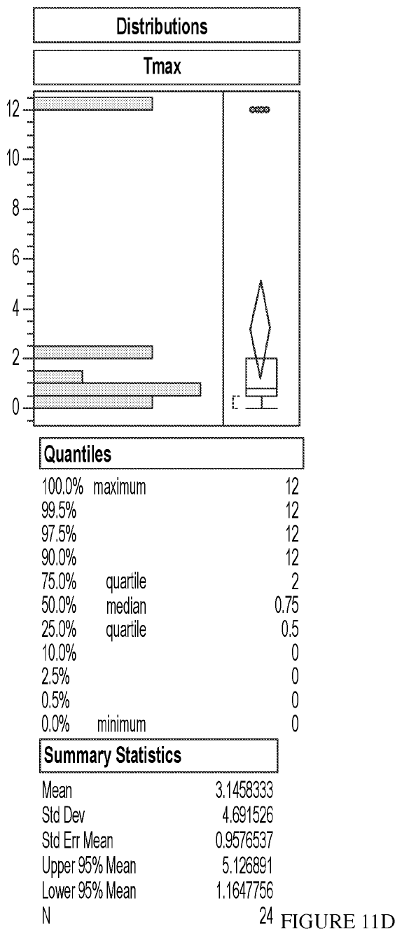

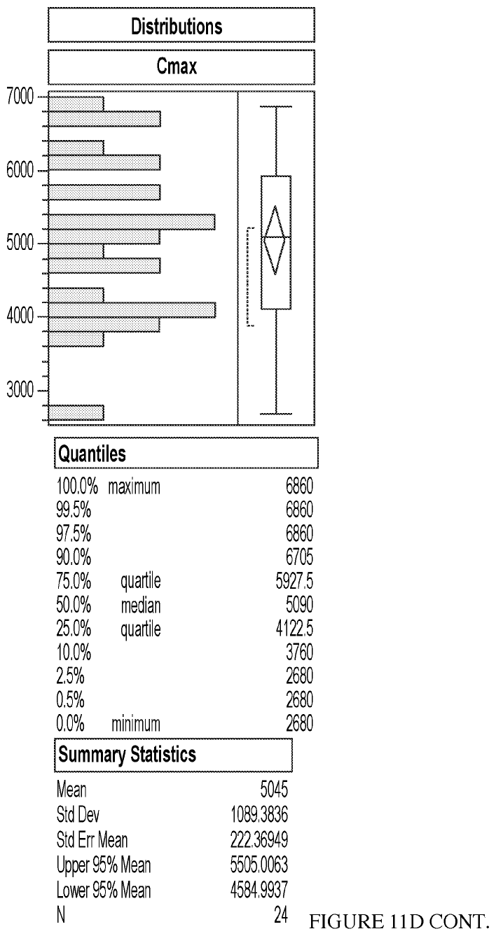

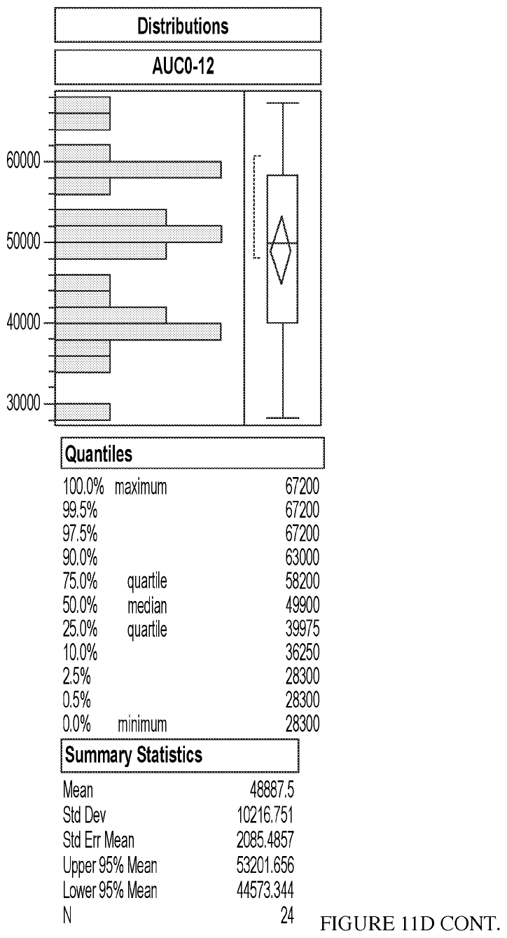

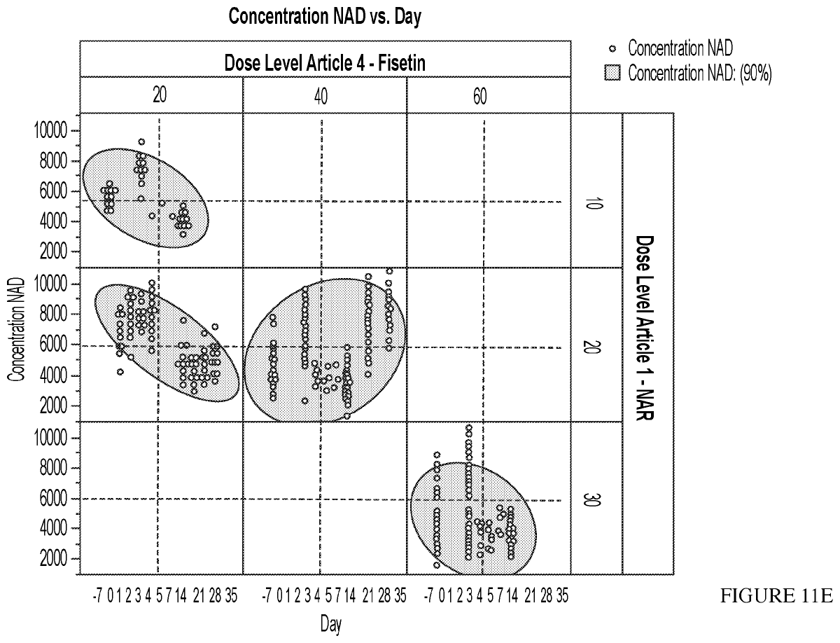

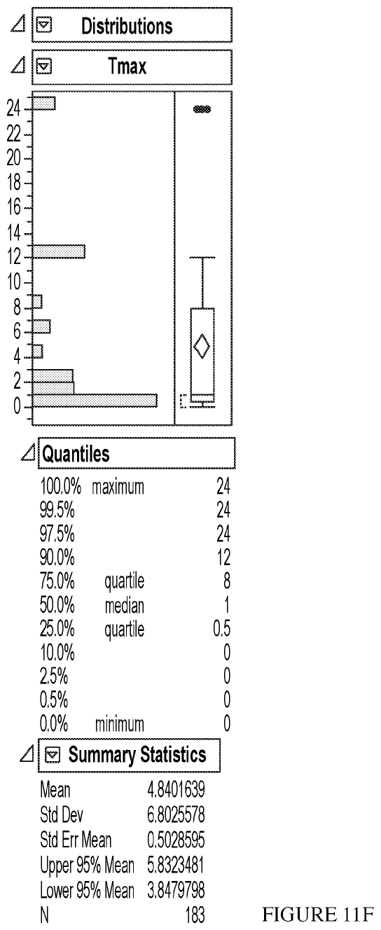

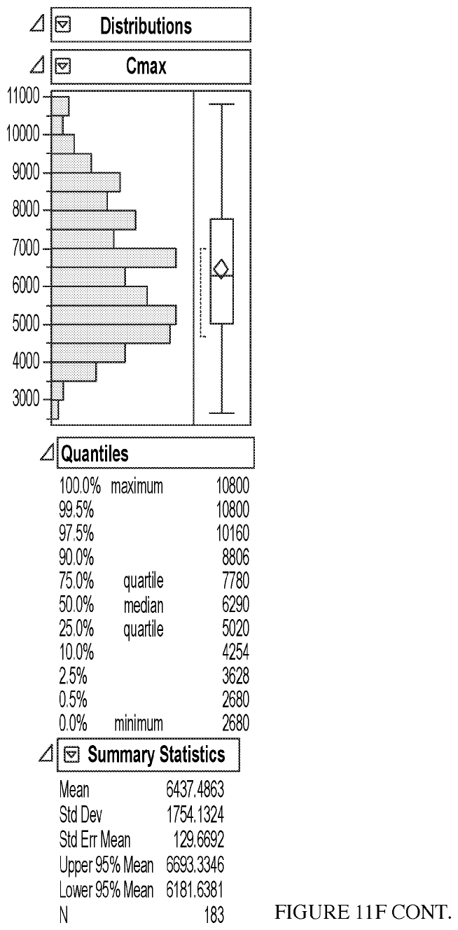

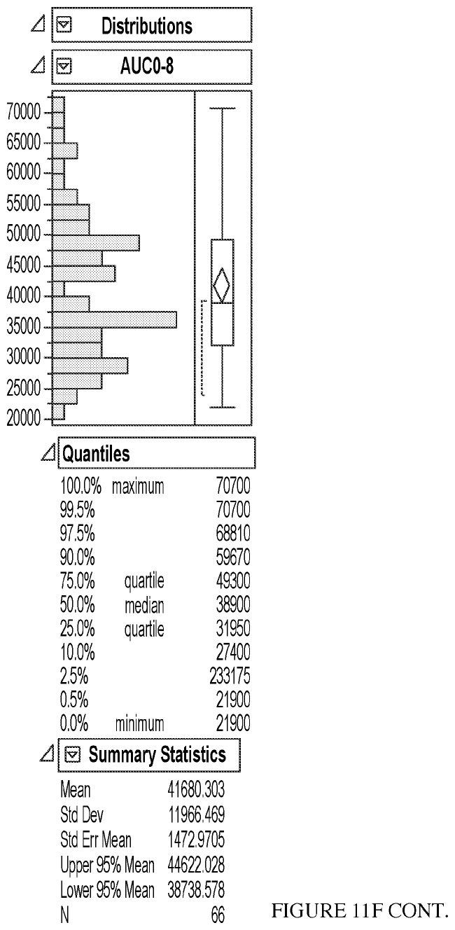

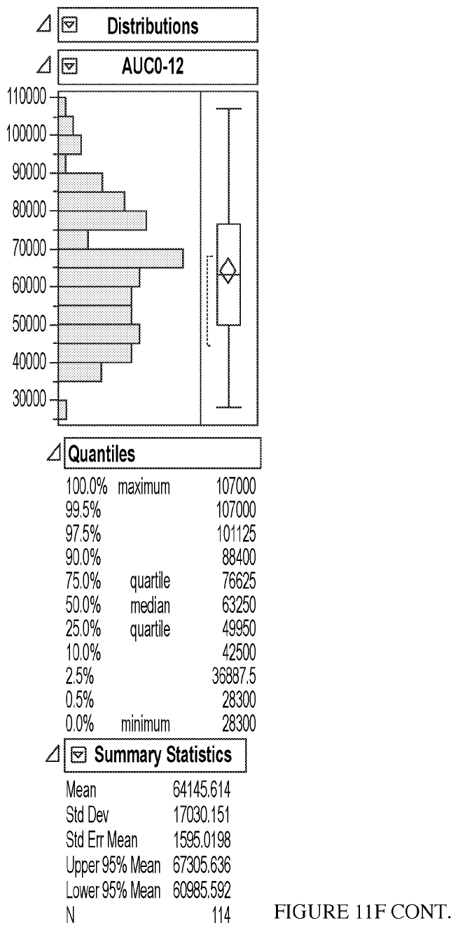

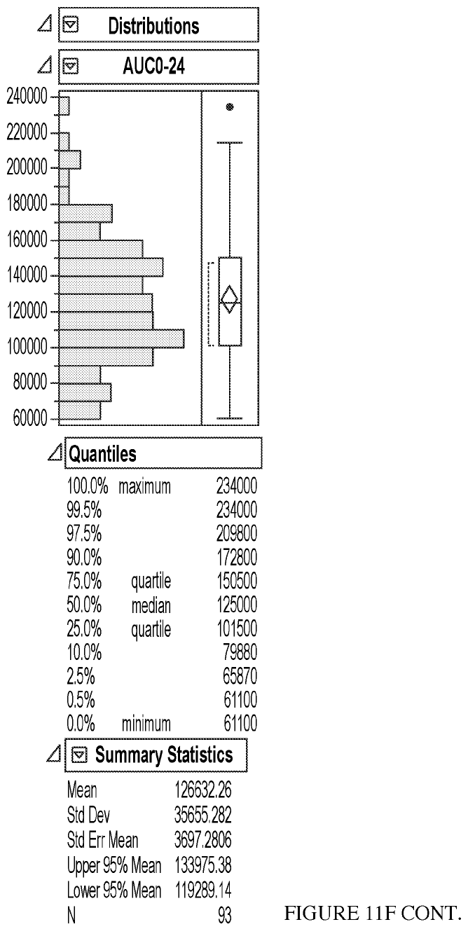

FIGS. 11A-11F show the difference in NAD concentration (ng/mL) by day for all of the dosing regimen in both Study #1 and Study #2. In the NaR only dosing (FIGS. 11A and 11B), there is an increase in NAD concentration from Day 1 to Day 7 observed with all dose levels. Interestingly, the dose level 20 mg/kg was the only one to produce a decrease in NAD concentration from Day 7 to Day 28. The median Cₘₐₓ was 6990 ng/mL and the median AUC₀₋₂₄ was 133,000 ng/mL. In fisetin only dosing (FIGS. 11C and 11D), the NAD concentration decreased at days 7, 14 and 21, and returned to baseline at Day 28. The fisetin only administration had a median Cₘₐₓ of 5090 ng/mL, and a median AUC₀₋₁₂ of 49,900 ng/mL. All combined dosages of NaR and fisetin show a decrease in NAD concentrations at day 7 compared to baseline values (FIGS. 11E and 11F). The dosage of 20 mg/kg NaR and 40 mg/kg fisetin shows an increase in NAD concentrations again at Days 21 and 35. The median Cₘₐₓ was 6290 ng/mL and the median AUC₀₋₂₄ was 125,000 ng/mL. This result is further displayed in FIG. 12, a graphical depiction of the whole blood NAD concentration, comparing the administration of 20 mg/kg NaR and 20 mg/kg fisetin vs. 20 mg/kg NaR and 40 mg/kg fisetin from Study #2. The plot shows a decrease on NAD concentration on days 7 and 14 when NaR is administered with both 20 mg/kg and 40 mg/kg doses of fisetin. The NAD concentrations remain decreased at days 21 and 28 with the 20 mg/kg NAR and 20 mg/kg fisetin dosing regimen. However, the NAD concentration recovers to values above baseline on days 28 and 35 with the 20 mg/kg NaR and 40 mg/kg fisetin dosing regimen. The increase in NAD concentration paired with the high AUC₀₋₂₄ of the 20 mg/kg NaR and 40 mg/kg fisetin dosing regimen is the basis for the choice as the optimal dosage.

### Example 7 Effectiveness of Oral Administration of NaR and Fisetin

The objective of the study described in this example is to evaluate the effectiveness of oral administration of NaR and fisetin in aging dogs to stabilize or improve their owner perceived quality of life (QOL), activity and mobility levels, muscle mass and attention span.

### Materials and Methods

### Animals for Study:

Canis familaris (dog)—sixty non-breed specific dogs, age 10+ years, weight 8-40 kg, meeting the inclusion criteria.

Inclusion Criteria:
1) The absence of known comorbidities that might affect health significantly over the 6-month period studied. The health status is initially established by review of veterinary records from within the previous 6 months, with blood work within the last 6 months.
2) Ability to walk independently on a non-slip surface.
3) Cognitive function: mild dysfunction—scoring >14 and <34 on the CADES scale.
4) All dogs must be visual and still able to hear and respond to commands.

### Animal Observations:

Baseline data is collected at the start of the study including information about the dog and their environment. A physical examination is conducted including a complete blood count (CBC) chemistry panel and urine analysis.

An owner questionnaire is collected including: i) a simple 0-10 scale; ii) a modified CORQ questionnaire that assesses 4 domains—vitality, companionship, pain and mobility (Giuffrida et al., JAVMA, 252(9):1073-1083 (2018), which is hereby included by reference in its entirety); iii) a client specific outcome measure; and iv) a happiness assessment. The owners additionally complete surveys assessing: i) activity—the Liverpool Osteoarthritis in Dogs (LOAD) survey; ii) pain—the Canine Brief Pain Inventory; iii) sleep quality—the Sleep Survey; and iv) cognition/behavior—the CADES survey.

The animals are assessed by the investigators to obtain data on: i) the grade of osteoarthritis; ii) the spinal pain grade; iii) a body and muscle condition score; iv) a mobility score; v) walking speed and stride length; vi) a stability test; vii) activity monitor data; and viii) cognitive tests.

A bank PBMC is performed as an assessment of epigenetic markers of aging.

### Dosing Schedule:

Once these baseline data are collected, the dogs are randomized in blocks of 12 using a random number generator. Medications/placebo are provided in a form that hides their identity, labelled as A, B, and C.

Group A: NaR 10 mg/kg daily and Fisetin 20 mg/kg on two consecutive days monthly.

Group B: NaR 20 mg/kg daily and Fisetin 40 mg/kg on two consecutive days monthly.

Group C: Placebo administered daily.

### Data Collection:

All owner and investigator animal observations are repeated at 1, 3 and 6 months. The blood work and UA are performed at baseline, 1 month and 6 months. Serum, plasma and PBMC banking are performed at baseline and 6 months.

### Outcome Analysis:

The primary outcome analyses of the study include a comparison in the change in CADES score over time, the differences in activity over time, and changes in CORQ score over time between the groups.

The secondary outcome analyses of the study include a comparison in the changes in outcome data on quality of life, pain, activity, and sleep from the owner questionnaires between the groups. The cognitive testing generates continuous data that is compared between groups. The mobility, postural stability, body and muscle condition, spinal pain, osteoarthritis scores and thigh circumference assessments are also compared.

While the present disclosure has been described with reference to the specific embodiments thereof, it should be understood by those skilled in the art that various changes may be made, and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the disclosure.

1. A combination comprising,
a. a first formulation comprising an NAD+ agonist; and
b. a second formulation comprising a flavonoid.

2. The combination of claim 1, wherein the NAD+ agonist is an NAD+ precursor.

3. The combination of claim 2, wherein the NAD+ precursor comprises one or a combination of two or more of nicotinamide mononucleotide (NMN), nicotinamide riboside (NR), nicotinic acid riboside (NaR), ester derivatives of nicotinic acid riboside, nicotinic acid (niacin), ester derivatives of nicotinic acid, nicotinic acid mononucleotide (NaMN), ester derivatives of nicotinic acid mononucleotide, nicotinic acid adenine dinucleotide (NaAD), nicotinic acid adenine dinucleotide (NAAD), or a pharmaceutically acceptable salts, derivatives or prodrugs thereof.

4. The combination of any one of claims 1-3, wherein the first formulation comprises NaR or a pharmaceutically acceptable salt, derivative or prodrug thereof.

5. The combination of claim 4, wherein the first formulation comprises a salt of NaR.

6. The combination of claim 5, wherein the first formulation comprises an inorganic salt of NaR.

7. The combination of claim 6, wherein the inorganic salt of NaR comprises Li⁺, Na⁺, K⁺, Rb⁺, Cs⁺, Be²⁺, Mg²⁺, Ca²⁺, Sr²⁺, Zn²⁺ and Ba²⁺ or a combination thereof.

8. The combination of claim 5, wherein the first formulation comprises an amino acid salt of NaR.

9. The combination of claim 8, wherein the amino acid salt of NaR is selected from phenylalanine, isoleucine, tryptophan, lysine, asparagine, valine, aspartic acid, alanine, arginine and proline.

10. The combination of claim 8, wherein the amino acid salt of NaR is the tryptophan salt.

11. The combination of any one of claims 1-3, wherein the first formulation comprises NaMN or a pharmaceutically acceptable salt, derivative or prodrug thereof.

12. The combination of claim 11, wherein the first formulation comprises an inorganic salt of NaMN.

13. The combination of claim 12, wherein the inorganic salt of NaMN comprises Li⁺, Na⁺, K⁺, Rb⁺, Cs⁺, Be²⁺, Mg²⁺, Ca²⁺, Sr²⁺, Zn²⁺ and Ba²⁺ or a combination thereof.

14. The combination of claim 11, wherein the first formulation comprises an amino acid salt of NaMN.

15. The combination of claim 14, wherein the amino acid salt of NaMN is selected from the mono L-valine salt, the mono L-glutamine salt, the di L-valine salt, the di L-glutamine salt, the mono L-histidine salt, the di L-histidine salt, the mono L-arginine salt, the mono L-tryptophan salt, the di L-arginine salt, the di L-glutamic salt, the di L-tryptophan salt, the di L-threonine salt, the di L-lysine salt, the mono L-methionine salt, the di L-leucine salt and the di D-valine salt, or a combination thereof.

16. The combination of any one of claims 1-3, wherein the first formulation comprises NMN or a pharmaceutically acceptable salt, derivative or prodrug thereof.

17. The combination of claim 16, wherein the first formulation comprises an inorganic salt of NMN.

18. The combination of claim 17, wherein the inorganic salt of NMN comprises Li⁺, Na⁺, K⁺, Rb⁺, Cs⁺, Be²⁺, Mg²⁺, Ca²⁺, Sr²⁺, Zn²⁺ and Ba²⁺ or a combination thereof.

19. The combination of claim 16, wherein the first formulation comprises an amino acid salt of NMN.

20. The combination of claim 19, wherein the amino acid salt of NMN is selected from a mono L-valine salt, a mono L-tryptophan salt, a mono L-proline salt, or combination thereof.

21. The combination of any one of claims 1-3, wherein the first formulation comprises NA or a pharmaceutically acceptable salt, derivative or prodrug thereof.

22. The combination of claim 21, wherein the first formulation comprises an inorganic salt of NA.

23. The combination of claim 22, wherein the inorganic salt of NA comprises Li⁺, Na⁺, K⁺, Rb⁺, Cs⁺, Be²⁺, Mg²⁺, Ca²⁺, Sr²⁺, Zn²⁺ and Ba²⁺ salt or a combination thereof.

24. The combination of claim 21, wherein the first formulation comprises an amino acid salt of NA.

25. The combination of any one of the preceding claims, wherein the first formulation is suitable for oral administration.

26. The combination of claim 25, wherein the first formulation is in the form of a tablet or capsule.

27. The combination of any one of the preceding claims, wherein the first formulation comprises at least about 50 mg of the NAD+ agonist.

28. The combination of claim 27, wherein the first formulation comprises at least about 100 mg, 150 mg, 150 mg, 200 mg, 250 mg, 300 mg, 350 mg, 400 mg, 500 mg, 750 mg, 1000 mg, 1250 mg, 2500 mg, 3500 mg, 5000 mg or 7000 mg of the NAD+ agonist.

29. The combination of any of the preceding claims, wherein the second formulation comprises a flavonoid selected from fisetin; quercetin; rutin; quercitrin; catechin; gallocatechin; catechin 3-gallate; gallocatechin 3-gallate; epicatechin; epigallocatechin; epicatechin 3-gallate; epigallocatechin3-gallate; theaflavin-3-gallate; theaflavin-3′-gallate; theaflavin-3,3′-digallate; kaempferol; myricetin; galangin; isorhamnetin; pachypodol; rhamnazin; a pyranoflavonol; furanoflavonol; luteolin; resveratrol; apigenin; tangeritin; hesperetin; naringenin; eriodictyol; homoeriodictyol; analogs and derivatives thereof; as well as a combination of two or more thereof.

30. The combination of claim 29, wherein the second formulation comprises fisetin (3,3′,4′,7-tetrahydroxyflavone) or an analog, derivative or metabolite thereof.

31. The combination of claim 30, wherein the second formulation comprises glucuronidated, sulfated or methylated metabolite of fisetin.

32. The combination of claim 30, wherein second formulation comprises glucuronidated fisetin.

33. The combination of any one of the preceding claims, wherein the second formulation comprises a NAD+ agonist.

34. The combination of claim 33, wherein the NAD+ agonist is an NAD+ precursor.

35. The combination of claim 34, wherein the NAD+ precursor comprises one or a combination of two or more of nicotinamide mononucleotide (NMN), nicotinamide riboside (NR), nicotinic acid riboside (NaR), ester derivatives of nicotinic acid riboside, nicotinic acid (niacin), ester derivatives of nicotinic acid, nicotinic acid mononucleotide (NaMN), ester derivatives of nicotinic acid mononucleotide, nicotinic acid adenine dinucleotide (NaAD), nicotinic acid adenine dinucleotide (NAAD), or a pharmaceutically acceptable salts, derivatives or prodrugs thereof.

36. The combination of any one of the preceding claims, wherein the second formulation comprises NaR or a pharmaceutically acceptable salt, derivative or prodrug thereof.

37. The combination of claim 36, wherein the second formulation comprises a salt of NaR.

38. The combination of claim 37, wherein the second formulation comprises an inorganic salt of NaR.

39. The combination of claim 38, wherein the inorganic salt of NaR comprises Li⁺, Na⁺, K⁺, Rb⁺, Cs⁺, Be²⁺, Mg²⁺, Ca²⁺, Sr²⁺, Zn²⁺ and Ba²⁺ or a combination thereof.

40. The combination of claim 37, wherein the second formulation comprises an amino acid salt of NaR.

41. The combination of claim 40, wherein the amino acid salt of NaR is selected from phenylalanine, isoleucine, tryptophan, lysine, asparagine, valine, aspartic acid, alanine, arginine and proline.

42. The combination of claim 41, wherein the amino acid salt of NaR is the tryptophan salt.

43. The combination of any one of claims 1-35, wherein the second formulation comprises NaMN or a pharmaceutically acceptable salt, derivative or prodrug thereof.

44. The combination of claim 43, wherein the second formulation comprises an inorganic salt of NaMN.

45. The combination of claim 44, wherein the inorganic salt of NaMN comprises Li⁺, Na⁺, K⁺, Rb⁺, Cs⁺, Be²⁺, Mg²⁺, Ca²⁺, Sr²⁺, Zn²⁺ and Ba²⁺ or a combination thereof.

46. The combination of claim 43, wherein the second formulation comprises an amino acid salt of NaMN.

47. The combination of claim 46, wherein the amino acid salt of NaMN is selected from the mono L-valine salt, the mono L-glutamine salt, the di L-valine salt, the di L-glutamine salt, the mono L-histidine salt, the di L-histidine salt, the mono L-arginine salt, the mono L-tryptophan salt, the di L-arginine salt, the di L-glutamic salt, the di L-tryptophan salt, the di L-threonine salt, the di L-lysine salt, the mono L-methionine salt, the di L-leucine salt and the di D-valine salt, or a combination thereof.

48. The combination of any one of claims 1-35, wherein the second formulation comprises NMN or a pharmaceutically acceptable salt, derivative or prodrug thereof.

49. The combination of claim 48, wherein the second formulation comprises an inorganic salt of NMN.

50. The combination of claim 49, wherein the inorganic salt of NMN Li⁺, Na⁺, K⁺, Rb⁺, Cs⁺, Be²⁺, Mg²⁺, Ca²⁺, Sr²⁺, Zn²⁺ and Ba²⁺ or a combination thereof.

51. The combination of claim 48, wherein the second formulation comprises an amino acid salt of NMN.

52. The combination of claim 51, wherein the amino acid salt of NMN is selected from a mono L-valine salt, a mono L-tryptophan salt, a mono L-proline salt, or combination thereof.

53. The combination of any one of claims 1-35, wherein the second formulation comprises NA or a pharmaceutically acceptable salt, derivative or prodrug thereof.

54. The combination of claim 53, wherein the second formulation comprises an inorganic salt of NA.

55. The combination of claim 53, wherein the inorganic salt of NA comprises Li⁺, Na⁺, K⁺, Rb⁺, Cs⁺, Be²⁺, Mg²⁺, Ca²⁺, Sr²⁺, Zn²⁺ and Ba²⁺ salt or a combination thereof.

56. The combination of claim 53, wherein the second formulation comprises an amino acid salt of NA.

57. The combination of any one of claims 33-56, wherein the second formulation comprises fisetin.

58. The combination of any one of the preceding claims, wherein the second formulation is formulated for oral administration.

59. The combination of claim 58, wherein the second formulation is in the form of a tablet or capsule.

60. The combination of any one of the preceding claims, wherein the second formulation comprises at least about 100 mg, 150 mg, 150 mg, 200 mg, 250 mg, 300 mg, 350 mg, 400 mg, 500 mg, 750 mg, 1000 mg, 1250 mg, 2500 mg, 3500 mg, 5000 mg or 7000 mg of the flavonoid.

61. The combination of any one of the preceding claims, wherein the second formulation comprises at least about 50 mg of the NAD+ agonist.

62. The combination of claim 58, wherein the second formulation comprises at least about 100 mg, 150 mg, 150 mg, 200 mg, 250 mg, 300 mg, 350 mg, 400 mg, 500 mg, 750 mg, 1000 mg, 1250 mg, 2500 mg, 3500 mg, 5000 mg or 7000 mg of the NAD+ agonist.

63. The combination of claim 1, wherein the first formulation comprises NaR or a pharmaceutically acceptable salt thereof.

64. The combination of claim 1, wherein the first formulation comprises a NaR or a pharmaceutically acceptable salt thereof and the second formulation comprises fisetin.

65. The combination of claim 1, wherein the first formulation comprises an inorganic salt of NaR and the second formulation comprises fisetin.

66. The combination of claim 1, wherein the first formulation comprises an amino acid salt of NaR and the second formulation comprises fisetin.

67. The combination of claim 1, wherein the first formulation comprises the tryptophan salt of NaR and the second formulation comprises fisetin.

68. The combination of claim 1, wherein the first formulation comprises NaMN or a pharmaceutically acceptable salt thereof and the second formulation comprises fisetin.

69. The combination of claim 1, wherein the first formulation comprises an inorganic salt of NaMN and the second formulation comprises fisetin.

70. The combination of claim 1, wherein the first formulation comprises an amino acid salt of NaMN and the second formulation comprises fisetin.

71. The combination of claim 1, wherein the first formulation comprises the tryptophan salt of NaMN and the second formulation comprises fisetin.

72. The combination of claim 1, wherein the first formulation comprises NaR or a pharmaceutically acceptable salt thereof and the second formulation comprises fisetin and NaR or a pharmaceutically acceptable salt thereof.

73. The combination of claim 1, wherein the first formulation comprises an inorganic salt of NaR and the second formulation comprises fisetin and an inorganic salt of NaR.

74. The combination of claim 1, wherein the first formulation comprises an amino acid salt of NaR and the second formulation comprises fisetin and an amino acid salt of NaR.

75. The combination of claim 1, wherein the first formulation comprises the tryptophan salt of NaR and the second formulation comprises fisetin and the tryptophan salt of NaR.

76. The combination of claim 1, wherein the first formulation comprises NaMN or pharmaceutically acceptable salt thereof and the second formulation comprises fisetin and NaMN or a pharmaceutically acceptable salt thereof.

77. The combination of claim 1, wherein the first formulation comprises an inorganic salt of NaMN and the second formulation comprises fisetin and an inorganic salt of NaMN.

78. The combination of claim 1, wherein the first formulation comprises an amino acid salt of NaMN and the second formulation comprises fisetin and an amino acid salt of NaMN.

79. The combination of claim 1, wherein the first formulation comprises the tryptophan salt of NaMN and the second formulation comprises fisetin and the tryptophan salt of NaMN.

80. A kit or article of manufacture comprising the combination of any one of the preceding claims.

81. A method of treating or preventing an age-related disorder associated with aging comprising administering to a subject in need thereof the combination of any one of claims 1-79.

82. A method of treating or preventing a neurodegenerative disease or disorder comprising administering to a subject in need thereof the combination of any one of claims 1-79.

83. The method of claim 81 or claim 82, wherein the NAD+ agonist is administered at a dose of between about 10 to about 100 mg/kg.

84. The method of claim 83, wherein the NAD+ agonist is administered at a dose of about 20 mg/kg.

85. The method of claim 83 or claim 84, wherein the NAD+ agonist comprises NaR or a pharmaceutically acceptable salt, derivative or prodrug thereof.

86. The method of claim 83 or claim 84, wherein the NAD+ agonist comprises NMN or a pharmaceutically acceptable salt, derivative or prodrug thereof.

87. The method of any one of claims 81-86, wherein the flavonoid is administered at a dose of between about 20 mg/kg to about 100 mg/kg.

88. The method of claim 87, wherein the flavonoid is administered at a dose of about 40 mg/kg.

89. The method of claim 87 or claim 88, wherein the flavonoid is fisetin.

90. The method of any one of claims 81-89, wherein the NAD+ agonist is administered daily.

91. The method of any one of claims 81-90, wherein the flavonoid is administered at least twice per week.

92. The method of any one of claims 81-90, wherein the flavonoid is administered at least three times per week.

93. The method of any one of claims 81-90, wherein the flavonoid is administered for 2-3 consecutive days per week.

94. The method of any one of claims 81-90, wherein the flavonoid is administered for 2-3 consecutive days every other week.

95. The method of any one of claims 81-90, wherein the flavonoid is administered at least twice per month.

96. The method of any one of claims 81-90, wherein the flavonoid is administered for 2-3 consecutive day per month.

97. The method of any one of claims 81-90, wherein the flavonoid is administered for 2 consecutive day per month.

98. The method of any one of claims 81-90, comprising a weekly dosage regimen wherein the dosage regimen comprises administration of the second formulation of any one of claims 33-62 and 72-79 for at least 2-3 consecutive days followed by daily administration of the first formulation.

99. The method of any one of claims 81-90, comprising a monthly dosage regimen wherein the dosage regimen comprises administration of the second formulation of any one of claims 33-62 and 72-79 for least 2-3 consecutive days every other week and daily administration of the first formulation on the remaining days of the month.

100. The method of any one of claims 81-84, comprising a monthly dosage regimen wherein the dosage regimen comprises:
daily administration of the first formulation, wherein the NAD+ agonist is NaR or a pharmaceutically acceptable salt, derivative or prodrug thereof, at a dose of about 20 mg/kg; and
administering for two consecutive days the second formulation, wherein the flavonoid is fisetin, at a dose of about 40 mg/kg.

101. The method of any one of claims 81-84, comprising a monthly dosage regimen wherein the dosage regimen comprises:
daily administration of the first formulation, wherein the NAD+ agonist is NMN or a pharmaceutically acceptable salt, derivative or prodrug thereof, at a dose of about 20 mg/kg; and
administering for two consecutive days the second formulation, wherein the flavonoid is fisetin, at a dose of about 40 mg/kg.

102. The method of claim 100 or claim 101, wherein the monthly dosing regimen is 28 days in length, and wherein the administering of the second formulation occurs on the first and second days of the 28 days.

103. The method of claim 102, wherein the monthly dosing regimen is repeated for at least 1 month, at least 2 months, at least 3 months, at least 4 months, at least 5 months, at least 6 months, at least 7 months, at least 8 months, at least 9 months, at least 10 months, at least 11 months, at least 12 months, at least 24 months, at least 36 months, at least 48 months, at least 60 months, at least 72 months, at least 84 months, at least 96 months, at least 108 months, at least 120 months, or at least 240 months.

104. The method of any one of claims 81 and 83-103, wherein the age related disease or disorder is selected form loss of eye function, reduction in bone density, insulin insensitivity, frailty, osteoarthritis, cognitive dysfunction (e.g., dementia), metabolic disease, obesity, inflammatory syndromes, cardiovascular disease, lipid disorders, age related muscle loss, osteoporosis, chronic kidney disease, vascular disease, or a combination thereof.

105. The method of any one of claims 82-103, wherein the neurodegenerative disease or disorder is Lewy body diseases, Alzheimer's disease, amnio tropic lateral sclerosis (ALS), Parkinson's Disease, Huntington's Chorea, senile dementia, Pick's disease, parkinsonism dementia syndrome, progressive subcortical gliosis, progressive supranuclear palsy, thalamic degeneration syndrome, hereditary aphasia, or myo-clonus epilepsy.

106. The method of any one of claims 82-103, wherein the neurodegenerative disease or disorder is frontal-temporal dementia, mood and anxiety disorders, depression, Schizophrenia, autism, anxiety, panic attacks, binge eating, social phobia, an affective disorder, a psychiatric disorder, mild cognitive impairment, seizures, neurodegenerative illnesses, dementia, head trauma or injury, hysteria accompanied by confusion, cognitive disorders; age-related dementia; age-induced memory impairment; ion deficit disorder; psychosis; cognitive deficits associated with psychosis.
